(19) United States
(12) Patent Application Publication
(10) Pub. No.: US 2020/0138347 A1
(43) Pub. Date: May 7, 2020

(54) DEVICES FOR BIOFLUID SAMPLE CONCENTRATION

(71) Applicants: Eccrine Systems, Inc., Cincinnati, OH (US); University of Cincinnati, Cincinnati, OH (US)

(72) Inventor: Jason C. Heikenfeld, Cincinnati, OH (US)

(73) Assignees: Eccrine Systems, Inc., Cincinnati, OH (US); University of Cincinnati, Cincinnati, OH (US)

(21) Appl. No.: 16/722,953

(22) Filed: Dec. 20, 2019

Related U.S. Application Data
(63) Continuation-in-part of application No. 15/770,262, filed on Apr. 23, 2018.
(60) Provisional application No. 62/269,244, filed on Dec. 18, 2015.
(60) Provisional application No. 62/269,447, filed on Dec. 18, 2015.
(60) Provisional application No. 62/245,638, filed on Oct. 23, 2015.
(60) Provisional application No. 62/783,273, filed on Dec. 21, 2018.

(51) Int. Cl. A61B 5/145; A61B 5/1468

(52) U.S. Cl. CPC A61B 5/14546; A61B 5/1468; A61B 5/14517; A61B 5/1451; A61B 5/14514

(57) ABSTRACT

The disclosed invention provides a fluid sensing device capable of collecting a biofluid sample, such as interstitial fluid, blood, sweat, or saliva, concentrating the sample with respect to a target analyte, and measuring the target analyte in the concentrated sample. Embodiments of the invention can also determine the change in molarity of the fluid sample with respect to the target analyte, as the sample is concentrated by the device. Some embodiments of the disclosed invention provide a fluid sensing device comprising minimally invasive, microneedle-enabled extraction of interstitial fluid or other biofluid for continuous or prolonged on-body monitoring of biomarkers. Some embodiments allow the collection and measurement of analytes in of non-biological fluids, such as fuels, or bodies of water.

### CROSS-REFERENCE TO RELATED APPLICATIONS

This application is a Continuation-in-Part of U.S. application Ser. No. 15/770,262, filed Apr. 23, 2018, and claims priority to PCT/US16/58356, filed Oct. 23, 2016; U.S. Provisional No. 62/783,273, filed Dec. 21, 2018; U.S. Provisional No. 62/245,638, filed Oct. 23, 2015; U.S. Provisional No. 62/269,244, filed Dec. 18, 2015, and U.S. Provisional No. 62/269,447, filed Dec. 18, 2015, the disclosures of which are hereby incorporated by reference herein in their entirety.

### BACKGROUND OF THE INVENTION

Non-invasive biosensing technologies have enormous potential for several medical, fitness, and personal well-being applications. The sweat ducts can provide a route of access to many of the same biomarkers, chemicals, or solutes that are carried in blood and can provide significant information enabling one to diagnose ailments, health status, toxins, performance, and other physiological attributes even in advance of any physical sign. Sweat has many of the same analytes and analyte concentrations found in blood and interstitial fluid. Interstitial fluid has even more analytes nearer to blood concentrations than sweat does, especially for larger sized and more hydrophilic analytes (such as proteins).

While bio-monitoring fluids offer their greatest potential when used as a source of continuous information about the body, the technological challenges of accomplishing such continuous monitoring are considerable. For example, many techniques that work well in a laboratory are difficult to implement in a wearable device. This is especially true for laboratory techniques used to measure analytes that typically emerge in sweat, interstitial fluid, or other fluid below the detection limit for available sensors. To overcome this challenge, devices and methods for concentrating fluid samples inside a wearable device are needed, and disclosed herein.

### SUMMARY OF THE INVENTION

The disclosed invention provides a fluid sensing device capable of collecting a biofluid sample, such as interstitial fluid, blood, sweat, or saliva, concentrating the sample with respect to a target analyte, and measuring the target analyte in the concentrated sample. Embodiments of the invention can also determine the change in molarity of the fluid sample with respect to the target analyte, as the sample is concentrated by the device. Some embodiments of the disclosed invention provide a fluid sensing device comprising minimally invasive, microneedle-enabled extraction of interstitial fluid or other biofluid for continuous or prolonged on-body monitoring of biomarkers. Some embodiments allow the collection and measurement of analytes in of non-biological fluids, such as fuels, or bodies of water.

### BRIEF DESCRIPTION OF THE DRAWINGS

The objects and advantages of the present disclosure will be further appreciated in light of the following detailed descriptions and drawings in which:

FIG. 1 is a depiction of at least a portion of a wearable device for biofluid sensing.

FIG. 2 is an example embodiment of at least a portion of a device capable of fluid sample concentration.

FIGS. 3A and 3B is an example embodiment of at least a portion of a device capable of fluid sample concentration.

FIG. 4 is an example embodiment of at least a portion of a device capable of fluid sample concentration.

FIG. 5 is an example embodiment of at least a portion of a device capable of fluid sample concentration.

FIG. 6 is an example embodiment of at least a portion of a device capable of fluid sample concentration.

FIG. 7 is an illustrated data plot of how the disclosed invention could be utilized.

FIG. 8 depicts an example embodiment of at least a portion of a device capable of fluid sample concentration.

FIG. 9 depicts an example embodiment of at least a portion of a device capable of fluid sample concentration.

FIG. 10 depicts an example embodiment of at least a portion of a device capable of fluid sample concentration.

FIG. 11A depicts an example embodiment of at least a portion of a device capable of fluid sample concentration.

FIG. 11B depicts a plan diagram of at least a portion of a device capable of fluid sample concentration.

FIGS. 12A to 12C depict example embodiments of at least a portion of a device capable of fluid sample concentration.

FIG. 13A depicts an example embodiment of at least a portion of a device capable of fluid sample concentration, and which is additionally capable of sweat stimulation and/or reverse iontophoresis.

FIGS. 13B to 13D depict plan diagrams of at least a portion of a device capable of fluid sample concentration.

FIGS. 14A and 14B depict example embodiments of at least a portion of a device capable of fluid sample concentration.

FIGS. 15A and 15B depict an example embodiment of at least a portion of a device capable of fluid sample concentration.

FIG. 16 depicts an example embodiment of at least a portion of a device capable of fluid sample concentration.

FIG. 17 depicts an example embodiment of at least a portion of a device capable of fluid sample concentration having at least one microneedle for extracting biofluid.

FIG. 18 depicts an example embodiment of at least a portion of a device capable of fluid sample concentration having at least one microneedle for extracting biofluid.

FIG. 19 depicts an example embodiment of at least a portion of a device capable of fluid sample concentration having at least one microneedle for extracting biofluid.

FIG. 20 depicts an example embodiment of at least a portion of a device capable of fluid sample concentration having at least one microneedle for extracting biofluid.

FIG. 21 depicts an example embodiment of at least a portion of a device capable of concentrating a biofluid sample extracted through a perforation in skin.

FIG. 22 depicts an example embodiment, similar to FIG. 13A, of at least a portion of a device capable of concentrating a biofluid sample, and further capable of electroosmosis and/or reverse iontophoresis.

### DEFINITIONS

“Analyte” means a substance, molecule, ion, or other material that is measured by a biofluid sensing device.

As used herein, “sweat” or “sweat biofluid” means a biofluid that is primarily sweat, such as eccrine or apocrine sweat, and may also include mixtures of biofluids such as sweat and blood, or sweat and interstitial fluid, so long as advective transport of the biofluid mixtures (e.g., flow) is primarily driven by sweat.

As used herein, “biofluid” may mean any human biofluid, including, without limitation, sweat, interstitial fluid, blood, plasma, serum, tears, and saliva. A biofluid may be diluted with water or other solvents inside a device because the term biofluid refers to the state of the fluid as it emerges from the body.

As used herein, “interstitial fluid” is a solution that bathes and surrounds tissue cells. The interstitial fluid is found in the interstices between cells. Embodiments of the disclosed invention measure analytes from interstitial fluid found in the skin and, particularly, interstitial fluid found in the dermis. In some cases where interstitial fluid is emerging from sweat ducts, the interstitial fluid contains some sweat as well, or alternately, sweat may contain some interstitial fluid.

As used herein, “fluid” may mean any human biofluid, or other fluid, such as water, including without limitation, groundwater, sea water, freshwater, wastewater, fuels, biofluels, etc., or other fluids.

As used herein, “continuous monitoring” means the capability of a device to provide at least one sensing and measurement of fluid collected continuously or on multiple occasions, or to provide a plurality of fluid measurements over time.

As used herein, “chronological assurance” is an assurance of the sampling rate for measurement(s) of sweat, interstitial fluid (or other biofluid or fluid), or solutes in biofluid, being the rate at which measurements can be made of new biofluid or its new solutes as they originate from the body. Chronological assurance may also include a determination of the effect of sensor function, or potential contamination with previously generated biofluid, previously generated solutes, other fluid, or other measurement contamination sources for the measurement(s).

As used herein, “determined” may encompass more specific meanings including but not limited to: something that is predetermined before use of a device; something that is determined during use of a device; something that could be a combination of determinations made before and during use of a device.

As used herein, “measured” can imply an exact or precise quantitative measurement and can include broader meanings such as, for example, measuring a relative amount of change of something. Measured can also imply a binary measurement, such as ‘yes’ or ‘no’ type qualitative measurements.

As used herein, “biofluid sampling rate” or “sampling rate” is the effective rate at which new biofluid, originating from pre-existing pathways, reaches a sensor that measures a property of the fluid or its solutes. Sampling rate is the rate at which new biofluid is refreshed at the one or more sensors and therefore old biofluid is removed as new fluid arrives. In one embodiment, this can be estimated based on volume, flow-rate, and time calculations, although it is recognized that some biofluid or solute mixing can occur. Sampling rate directly determines or is a contributing factor in determining the chronological assurance. Times and rates are inversely proportional (rates having at least partial units of 1/seconds), therefore a short or small time required to refill sample volume can also be said to have a fast or high sampling rate. The inverse of sampling rate (1/s) could also be interpreted as a “sampling interval(s)”. Sampling rates or intervals are not necessarily regular, discrete, periodic, discontinuous, or subject to other limitations. Like chronological assurance, sampling rate may also include a determination of the effect of potential contamination with previously generated biofluid, previously generated solutes (analytes), other fluid, or other measurement contamination sources for the measurement(s). Sampling rate can also be in part determined from solute generation, transport, advective transport of fluid, diffusion transport of solutes, or other factors that will impact the rate at which new sample will reach a sensor and/or is altered by older sample or solutes or other contamination sources.

As used herein, “sweat stimulation” is the direct or indirect causing of sweat generation by any external stimulus, the external stimulus being applied for the purpose of stimulating sweat. Sweat stimulation, or sweat activation, can be achieved by known methods. For example, sweat stimulation can be achieved by simple thermal stimulation, chemical heating pad, infrared light, by orally administering a drug, by intradermal injection of drugs such as carbachol, methylcholine or pilocarpine, and by dermal introduction of such drugs using iontophoresis. A device for iontophoresis may, for example, provide direct current and use large lead electrodes lined with porous material, where the positive pole is dampened with 2% pilocarpine hydrochloride and the negative one with 0.9% NaCl solution. Sweat can also be controlled or created by asking the device wearer to enact or increase activities or conditions that cause them to sweat. These techniques may be referred to as active control of sweat generation rate.

As used herein, “sample generation rate” is the rate at which biofluid is generated by flow through pre-existing pathways. Sample generation rate is typically measured by the flow rate from each pre-existing pathway in nL/min/pathway. In some cases, to obtain total sample flow rate, the sample generation rate is multiplied by the number of pathways from which the sample is being sampled. Similarly, as used herein, “analyte generation rate” is the rate at which solutes move from the body or other sources toward the sensors.

As used herein, “fluid sampling rate” is the effective rate at which new fluid, or fluid solutes, originating from the fluid source, reaches a sensor that measures a property of the fluid or its solutes. Fluid sampling rate directly determines, or is a contributing factor in determining, the chronological assurance. Times and rates are inversely proportional (rates having at least partial units of 1/seconds), therefore a short or small time required to refill a fluidic volume can also be said to have a fast or high fluid sampling rate. The inverse of fluid sampling rate (1/s) could also be interpreted as a “fluid sampling interval(s)”. Fluid sampling rates or intervals are not necessarily regular, discrete, periodic, discontinuous, or subject to other limitations. Like chronological assurance, fluid sampling rate may also include a determination of the effect of potential contamination with previously generated fluid, previously generated solutes, other fluid, or other measurement contamination sources for the measurement(s). Fluid sampling rate can also be in whole or in part determined from solute generation, transport, advective transport of fluid, diffusion transport of solutes, or other factors that will impact the rate at which new fluid or fluid solutes reach a sensor and/or are altered by older fluid or solutes or other contamination sources. Sensor response times may also affect sampling rate.

As used herein, “sample volume” is the fluidic volume in a space that can be defined multiple ways. Sample volume may be the volume that exists between a sensor and the point of generation of a biofluid sample. Sample volume can include the volume that can be occupied by sample fluid between: the sampling site on the skin and a sensor on the skin where the sensor has no intervening layers, materials, or components between it and the skin; or the sampling site on the skin and a sensor on the skin where there are one or more layers, materials, or components between the sensor and the sampling site on the skin.

As used herein, “solute generation rate” is simply the rate at which solutes move from the body or other sources into a fluid. “Solute sampling rate” includes the rate at which these solutes reach one or more sensors.

As used herein, “microfluidic components” are channels in polymer, textiles, paper, or other components known in the art of microfluidics for guiding movement of a fluid or at least partial containment of a fluid.

As used herein, “state void of fluid” means a fluid sensing device component, such as a space, material or surface, that can be wetted, filled, or partially filled by fluid, when the component is entirely or substantially (e.g., >50%) dry or void of fluid.

As used herein, “advective transport” is a transport mechanism of a substance, or conserved property by a fluid, that is due to the fluid's bulk motion.

As used herein, “diffusion” is the net movement of a substance from a region of high concentration to a region of low concentration. This is also referred to as the movement of a substance down a concentration gradient.

As used herein, a “sample concentrator” or “concentrator” is any portion of a device, material, subsystem, or other component that can be utilized to increase the molarity of at least one fluid analyte, at least in part by removing a portion of the water that was originally with the at least one analyte when it exited the body.

“EAB sensor” means an electrochemical aptamer-based biosensor that is configured with multiple aptamer sensing elements that, in the presence of a target analyte in a fluid sample, produce a signal indicating analyte capture, and which signal can be added to the signals of other such sensing elements, so that a signal threshold may be reached that indicates the presence or concentration of the target analyte. Such sensors can be in the forms disclosed in U.S. Pat. Nos. 7,803,542 and 8,003,374 (the “Multi-capture Aptamer Sensor” (MCAS)), or in U.S. Provisional Application No. 62/523,835 (the “Docked Aptamer Sensor” (DAS)).

As used herein, the term “analyte-specific sensor” is a sensor specific to an analyte and performs specific chemical recognition of the analyte's presence or concentration (e.g., ion-selective electrodes, enzymatic sensors, electrochemical aptamer-based sensors, etc.). For example, sensors that sense impedance or conductance of a fluid, such as sweat, are excluded from the definition of analyte-specific sensor because sensing impedance or conductance merges measurements of all ions in sweat (i.e., the sensor is not chemically selective; it provides an indirect measurement). Sensors could also be optical, mechanical, or use other physical/chemical methods which are specific to a single analyte. Further, multiple sensors can each be specific to one of multiple analytes.

“Wicking pressure,” “wicking force,” “capillary pressure,” or “capillary force,” means a pressure or force that should be interpreted according to its general scientific meaning. For example, a capillary (tube) geometry can be said to have a capillary pressure or a wicking pressure. Or a wicking textile or gel may have a capillary pressure, even if the material is not geometrically a tube or a channel. Conversely, a wicking fiber can have an effective capillary pressure. Similarly, the (relatively empty) space between a material placed on skin and the skin surface can have an effective wicking pressure. The terms wicking or capillary pressure and wicking or capillary force may be used interchangeably herein to describe the effective pressure provided by any component or material that is capable of capturing biofluid by a negative pressure (i.e., pulling it into or along said component or material). For simplicity, the term “wicking pressure” will be used herein to refer to any of the above alternate terms. Wicking pressure also must be considered in its specific context, for example, if a sponge is fully saturated with water, then it has no remaining wicking pressure. Wicking pressure must therefore be interpreted as described in the specification for a device during use, and not interpreted in isolation or in contexts other than the disclosed devices or use scenarios.

“Collector” or “Wicking collector” or means any component of the disclosed invention that supports the creation of, or sustains, a volume reduced pathway, or that is the wicking element that receives biofluid before a biofluid sensing device sensor and is on or adjacent to skin. A wicking collector can be a microfluidic component, a capillary material, a wrinkled surface, a textile, a gel, a coating, a film, or any other component that satisfies the general criteria of the present disclosure. A wicking collector may be part of the same component or material that serves other purposes (e.g., a wicking pump or a wicking coupler), and in such cases, the portion of said component or material that at least in part receives biofluid before the sensor(s) and is on or adjacent to skin is also a wicking collector as defined herein.

“Pump” or “wicking pump” refers to any component of the disclosed invention that supports creation of or sustains a volume reduced pathway, or that receives biofluid after a biofluid sensing device sensor and has a primary purpose of collecting excess fluid to allow sustained operation of the device. A wicking pump may also include an evaporative material or surface that is configured to remove excess biofluid by evaporation of water. A wicking pump may be part of the same component or material that serves other purposes (e.g., a wicking collector or a wicking coupler), and in such cases, the portion of said component or material that at least in part receives biofluid after the sensor(s), is also a wicking pump as defined herein. Pump may also reference alternate configurations, such as a small mechanical pump, or osmotic pressure across a membrane, so long as the pressure generated satisfies the requirements described herein.

“Wicking coupler” or “coupler” refers to any component of the disclosed invention that is on or adjacent to a biofluid sensing device sensor and that promotes coupling and transport of a biofluid or its solutes by advective flow, diffusion, or other method of transport, between another wicking component or material and at least one device sensor. In some embodiments, the coupler function may be performed by a suitably configured wicking collector. In other embodiments, a device sensor may be configured with a wicking surface or material that functions without a wicking coupler (such as an immobilized aptamer layer which is hydrophilic, or polymer ionophore layer which is porous to the analyte). A coupler may be part of the same component or material that serves other purposes (e.g., a wicking collector or a pump), and in such cases, the portion of said component or material that, at least in part, couples biofluid to a sensor(s) and that is on or adjacent to the sensor(s), is also a wicking coupler as defined herein.

“Wicking space” refers to the space between the skin and wicking collector that would be filled by air, skin oil, or other non-sweat fluids or gases if no sweat existed. In some embodiments of the disclosed invention, even if sweat exists, the wicking collector removes some or most of sweat from the wicking space by action of wicking pressure provided by the wicking collector.

As used herein, “pre-existing pathways” refer to pores, pathways, or routes through skin through which interstitial fluid may be extracted. Pre-existing pathways include but are not limited to: eccrine sweat ducts, other types of sweat ducts, hair follicles, inter-cell junctions, tape-stripping of the stratum comeum, skin defects, pathways created by electroporation of skin (e.g., of the stratum comeum), laser poration of skin, mechanical poration of skin (e.g., micro-needle rollers), chemical or solvent based poration of skin, or other methods or techniques. It should be recognized that “pre-existing” does not require that such pathways must be naturally occurring or that such pathways must exist prior to application of the device. Rather, methods of the disclosed invention may be practiced using a pathway that naturally exists or that was created for the particular application. Therefore, any technique to provide pre-existing pathways may be used in conjunction with embodiments of the disclosed invention. For example, a microneedle is a pre-existing pathway if the microneedle uses reverse iontophoresis for analyte extraction. As another example, electroporation of the lining of the sweat glands may form or affect a pre-existing pathway. As another example, skin permeability enhancing agents or chemicals may form part or all of a pre-existing pathway.

As used herein, “reverse iontophoresis” is a subset or more specific form of “iontophoresis” and is a technique by which electrical current and electrical field cause molecules to be removed from within the body by electro-osmosis and/or iontophoresis. Although the description below focuses primarily on electro-osmosis, the term “reverse iontophoresis” as used herein may also apply to flux of analytes brought to or into the devices of the disclosed invention, where the flux is in whole or at least in part due to iontophoresis (e.g., some negatively charged analytes may be transported against the direction of electro-osmotic flow and eventually onto a device according to an embodiment of the disclosed invention). Electro-osmotic flow (or electro-osmotic flow, synonymous with electro-osmosis or electro-endosmosis) is the motion of liquid induced by an applied potential across a porous material, capillary tube, membrane, microchannel, or any other fluid conduit. Because electro-osmotic velocities are independent of conduit size, as long as the electrical double layer is much smaller than the characteristic length scale of the channel, electro-osmotic flow is most significant when in small channels. In biological tissues, the negative surface charge of plasma membranes causes accumulation of positively charged ions such as sodium. Accordingly, fluid flow due to reverse iontophoresis in the skin is typically in the direction of where a negative voltage is applied (i.e., the advective flow of fluid is in the direction of the applied electric field). As used herein, the term “iontophoresis” may be substituted for “reverse iontophoresis” in any embodiment where there is a net advective transport of biofluid to the surface of the skin. For example, if a flow of sweat exists, then negatively charged analytes may be brought into the advectively flowing sweat by iontophoresis. The net advective flow of sweat would typically be needed, because in this case, a net electro-osmotic fluid flow would be in the direction of sweat into interstitial fluid (and without a net advective flow of sweat, the sweat would be lost, and there would be no pathway for transporting the analyte to at least one sensor). Furthermore, because “reverse iontophoresis” is a subset or more specific form of “iontophoresis”, the term “iontophoresis” may refer to both “reverse iontophoresis” and “iontophoresis”. The terms “reverse iontophoresis” and “iontophoresis” are interchangeable in the disclosed invention.

### DETAILED DESCRIPTION OF THE INVENTION

One skilled in the art will recognize that the various embodiments may be practiced without one or more of the specific details described herein, or with other replacement and/or additional methods, materials, or components. In other instances, well-known structures, materials, or operations are not shown or described in detail herein to avoid obscuring aspects of various embodiments of the invention. Similarly, for purposes of explanation, specific numbers, materials, and configurations are set forth herein in order to provide a thorough understanding of the invention. Furthermore, it is understood that the various embodiments shown in the figures are illustrative representations and are not necessarily drawn to scale.

Reference throughout this specification to “one embodiment” or “an embodiment” means that a particular feature, structure, material, or characteristic described in connection with the embodiment is included in at least one embodiment of the invention, but does not denote that they are present in every embodiment. Thus, the appearances of the phrases “in an embodiment” or “in another embodiment” in various places throughout this specification are not necessarily referring to the same embodiment of the invention. Further, “a component” may be representative of one or more components and, thus, may be used herein to mean “at least one.”

Certain embodiments of the invention show sensors as simple individual components. It is understood that many sensors require two or more electrodes, reference electrodes, or additional supporting technology or features that are not captured in the description herein. Sensors are preferably electrical in nature, but may also include optical, chemical, mechanical, or other known biosensing mechanisms. Sensors can be in duplicate, triplicate, or more, to provide improved data and readings. Sensors may be referred to by what the sensor is sensing, for example: a sweat sensor; an impedance sensor; a fluid volume sensor; a sweat generation rate sensor; and a solute generation rate sensor. Certain embodiments of the disclosed invention show sub-components of what would be fluid sensing devices with more sub-components needed for use of the device in various applications, which are obvious (such as a battery), and for purpose of brevity and focus on inventive aspects are not explicitly shown in the diagrams or described in the embodiments of the invention. As a further example, many embodiments of the invention could benefit from mechanical or other means known to those skilled in wearable devices, patches, bandages, and other technologies or materials affixed to skin, to keep the devices or sub-components of the skin firmly affixed to skin or with pressure favoring constant contact with skin or conformal contact with even ridges or grooves in skin, and are included within the spirit of the disclosed invention. The present application has specification that builds upon PCT/US13/35092, the disclosure of which is hereby incorporated herein by reference in its entirety.

The detailed description of the present invention will be primarily, but not entirely, limited to devices, methods and sub-methods using wearable biofluid sensing devices. Therefore, although not described in detail here, other essential steps which are readily interpreted from or incorporated along with the present invention shall be included as part of the disclosed invention. The disclosure provides specific examples to portray inventive steps, but which will not necessarily cover all possible embodiments commonly known to those skilled in the art. For example, the specific invention will not necessarily include all obvious features needed for operation. Several specific, but non-limiting, examples can be provided as follows. The invention includes reference to the article in press for publication in the journal IEEE Transactions on Biomedical Engineering, titled “Adhesive RFID Sensor Patch for Monitoring of Sweat Electrolytes”; the article published in the journal AIP Biomicrofluidics, 9 031301 (2015), titled “The Microfluidics of the Eccrine Sweat Gland, Including Biomarker Partitioning, Transport, and Biosensing Implications”; as well as PCT/US16/36038, and U.S. Provisional Application No. 62/327,408, each of which is included herein by reference in their entirety.

The disclosed invention applies at least to any type of fluid sensor device that measures fluid, fluid generation rate, fluid chronological assurance, its solutes, solutes that transfer into fluid from skin, tissue, or other source, a property of or things on the surface of skin, or properties or things beneath the skin. The invention applies to fluid sensing devices which can take on forms including patches, bands, straps, portions of clothing, wearables, or any suitable mechanism that reliably brings sweat stimulating, fluid collecting, and/or fluid sensing technology into intimate proximity with fluid as it is generated. Some embodiments of the invention utilize adhesives to hold the device near the skin, but devices could also be held by other mechanisms that hold the device secure against the skin, such as a strap or embedding in a helmet.

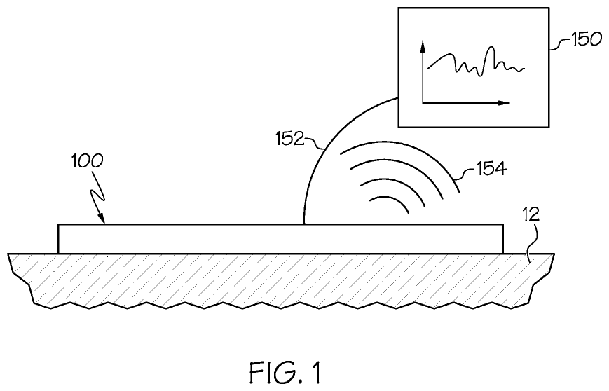

With reference to FIG. 1, a biofluid sensing device 100 is placed on or near skin 12. In an alternate embodiment, the biofluid sensing device may be fluidically connected to skin or regions near skin through microfluidics or other suitable techniques. Device 100 is in wired communication 152 or wireless communication 154 with a reader device 150. In one embodiment of the invention, the reader device 150 would be a smart phone or portable electronic device. In alternate embodiments, device 100 and reader device 150 can be combined. In further alternate embodiments, communication 152 or 154 is not constant and could be a simple one-time data download from device 100 once it has completed its measurements of biofluid.

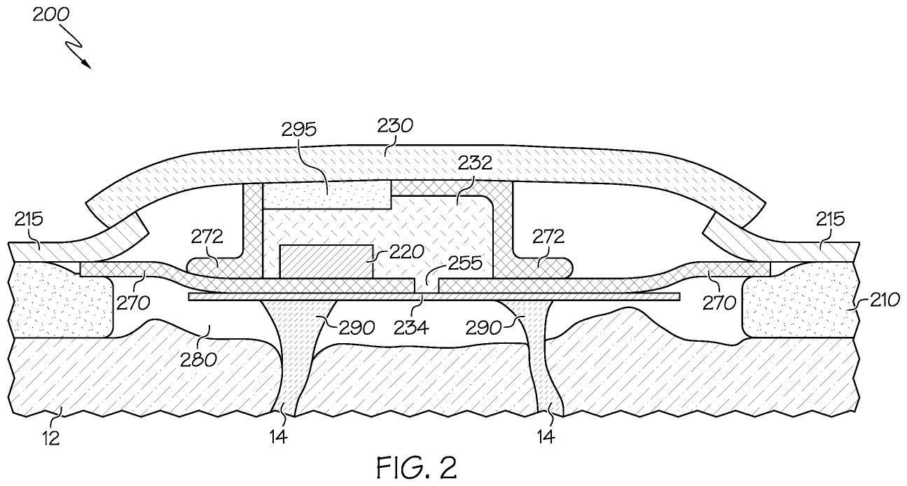

With reference to FIG. 2, a device 200 provides a reduced fluidic volume 280 between a wearer's skin 12 and at least one analyte-specific sensor 220, as disclosed in PCT/US2015/032893. The fluidic volume 280 is bounded by a fluid impermeable substrate 270 such as PET, and an adhesive layer 210, which also functions to secure the device to the skin. Material 270 has an opening in the center 255, to allow fluid to access the sensor 220. Adhesives can be pressure sensitive, liquid, tacky hydrogels, which promote robust electrical, fluidic, and iontophoretic contact with skin. The device 200 further includes fluid impermeable materials 215 and 272, where 215 may also serve as a substrate for fabrication (e.g., one or more layers shown in FIG. 2 could be fabricated on the substrate, for example a Kapton substrate for flexible electronics). The locations of sweat ducts 14 are also noted.

The device 200 is also configured to provide a reduced wicking volume, as disclosed in PCT US2016/43771. Accordingly, the device includes a sweat collector 234, which draws sweat through opening 255, and creates volume reduced pathway(s) 290 between the ducts and the opening 255. The sweat collector 234 is in fluidic communication with a fluid sample coupler 232, which carries sweat past the sensor 220. Sensor 220 could be any sensor specific to an analyte in sweat, such as an ion-selective electrode, enzymatic sensor, electrochemical aptamer sensor, etc. The fluid sample coupler 232 is in fluidic communication with a fluid sample pump 230, which is comprised of a textile, paper, or hydrogel, and that serves to maintain fluid flow through the device. The sweat collector 234 must be adequately thin so that its fluidic volume is less than the fluidic volume of the wicking space 280. As an example of a proper implementation of the sweat collector 234, the wicking space 280 could have an average height of 50 μm due to skin roughness, or more if hair or debris is present. The wicking material could be a 5 μm thick layer of screen-printed nanocellulose with a weak binder and or a thin hydrogel material to hold the cellulose together. Importantly, in terms of strength of capillary force, material 232 should have greater capillary force than material 230, which in turn should have greater capillary force than wicking space 280. In a preferred embodiment, fluid sample coupler 232 would have the greatest wicking force relative to the other wicking materials, such as 234 and 230, so that sensor 220 remains wetted with sweat.

With further reference to FIG. 2, the device 200 also includes a sample concentrator 295. In one embodiment of the invention, the sample concentrator 295 is a dialysis membrane that is permeable to inorganic ions but impermeable to small molecules and proteins. In other embodiments, the sample concentrator may be any membrane or material that is at least porous to water, but that is not substantially porous to the analyte that is to be concentrated. As sweat flows onto fluid sample pump 230 by wicking through the concentrator membrane 295, solutes are concentrated in fluid sample coupler 232. The device may be configured to concentrate a target analyte in the fluid sample by at least 2× higher than the unconcentrated molarity. Depending on the application, the target analyte may be concentrated at least 10×, 100×, or 1000× higher than the unconcentrated molarity. The fluid sample coupler 232 could be hydrogel, textile, or other suitable wicking material. Analyte-specific sensor 220 may be, for example, two or more electrochemical sensors for cortisol and dehydroepiandrosterone (DHEA) to measure the ratio of these two biomarkers in sweat. This ratio is a well-known marker of many conditions, see, e.g., http://metabolichealing.com/cortisol-dhea-the-major-hormone-balance, and furthermore allows meaningful sensing without having to determine the molarity of the fluid sample concentrated by the sample concentrator 295.

In an alternate embodiment, an osmosis membrane can be used as the sample concentrator 295, where the membrane is water-permeable, but is impermeable to electrolytes, such as K+. Because sweat K+ concentration does not vary significantly with sweat rate, the sensor 220 could measure K+ and another analyte, such as cortisol, to determine the molarity of the fluid sample, and therefore allow accurate back-calculation of the original cortisol concentration. Embodiments of the disclosed invention may accordingly be configured with a first sensor specific to a first fluid analyte and a second sensor specific to a second fluid analyte, wherein both the first and second analytes are concentrated. Similarly, additional sensors may be added to measure additional concentrated analytes. In other embodiments, sweat conductivity could be measured and used to determine the molarity, although this method would be less reliable, since sweat conductivity is more variable with sweat generation rate.

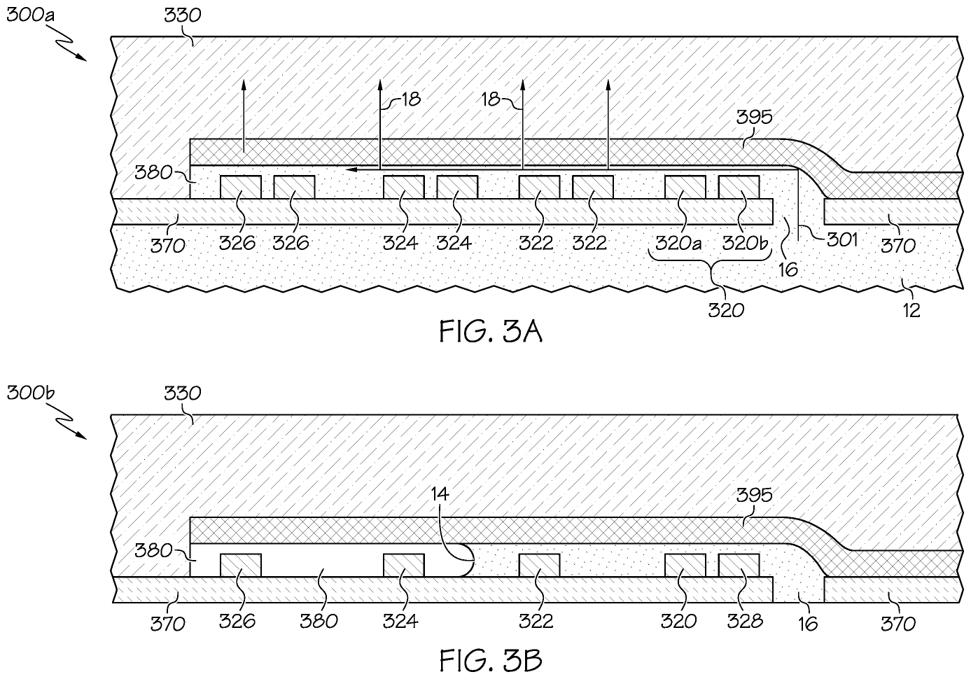

With reference to FIG. 3A, where like numerals refer to like features of previous figures, a channel 380 is created and filled with a fluid sample 16 between sample concentrator membrane 395 and fluid impermeable material or film 370. The channel 380 includes at least one pair of sensors 320a and 320b, that form a sensor pair 320. For example, a first sensor 320a may be for K+, and a second sensor 320b may be for cortisol. Sensor pair 320 measures the unconcentrated sweat concentrations of K+ and cortisol as they emerge from the skin 12. As fluid 16 flows leftward through the channel 380 in the direction of the arrow 301, water 18 from the fluid diffuses into the fluid sample pump 330, which may be a hydrogel, a desiccant, salt, or microfluidic wicking material, by osmosis or capillary wicking force. By this mechanism, the fluid sample 16 becomes more concentrated as it moves through the channel 380. Sensor pairs 322, 324, 326 likewise measure K+ and cortisol, but at increasing concentrations as the fluid sample 16 becomes more concentrated (i.e., loses more water through sample concentrator 395). The K+ sensor is used to predict the molarity of the sweat sample 16 and therefore correct the cortisol reading for the increasing molarity. One will recognize that the channel flow volume will decrease as the sample 16 moves through the channel 380 along the arrow 301, and in some cases the flow velocity or pressure will become too low to allow reliable measurement (e.g., diffusion or backflow will contaminate the sample). Therefore, in one example, the channel 380 may be tapered in at least one dimension along the channel length moving right to left. With such a configuration, as the fluid sample 16 flows from right to left, the sample's deceleration from fluid loss may be reduced, fluid velocity may remain steady, or fluid velocity may actually increase, as the volume of the channel decreases (as will be illustrated in a later figure). The sample concentrator membrane 395 could also have a surface charge in water, which would tend to reject permeation by ions such as K+. The invention may include a plurality of analyte-specific sensors for detecting at least one target analyte, wherein at least two of said plurality of sensors comprise a first sensor group, and at least two of said plurality of sensors comprise a second sensor group, and wherein the first sensor group measures the target analyte in a less concentrated fluid sample, and the second sensor group measures the target analyte in a more concentrated fluid sample.

With reference to FIG. 3B, the device 300b is similar to device 300a of FIG. 3A with several exceptions. As an initial matter, the sensors 320, 322, 324, 326 could be similar to those described previously, or may detect the presence of a fluid sample 16 by potential or conductance, where the fluid 16 is in its original, unconcentrated form, or in a concentrated form. A channel, tube, or other capillary component 380 is created between concentrator membrane 395 and water-impermeable film 370. Alternately, the channel could be formed entirely out of a tube of membrane material, such as used for the membrane 395 (e.g., a small dialysis tube). Multiple arrangements are possible, so long as they satisfy the general inventive aspects described herein. As shown, the advancing edge 14 of fluid 16 is concave and moving toward fluid sample pump 330. The fluid sample pump 330 could be a gel, a desiccant, and the membrane 395 could be membranes like previously taught for FIG. 2 or porous Teflon membrane filled with air, or other material which provides at least one gas filled pathway. Membrane 395 could therefore allow water to pass into the air as water vapor (evaporation). If evaporation is relied upon, then concentrator 395 may not need to be permi-selective to solutes in fluid, but rather would help define the fluidic channel 380. A fluid sample 16 could be collected, fill the channel, and water or solutes that are not of interest for sensing may be extracted through concentrator membrane 395. As water is lost, the fluid sample 16 reduces in volume, and the sensors 320, 322, 324, 326 can use a volume measurement to predict how much fluid sample 16 was collected initially and how much sample 16 has been concentrated. Therefore, the disclosed invention may include at least one sensor for volumetric measurement. Other sensor arrangements to determine the molarity of the sample 16 may also be possible, and the use of sensors 320, 322, 324, 326 are one non-limiting example.

In another example embodiment, sensor 328 may sense a target analyte, and the analyte's actual molarity can be calculated based on successive sensor 320, 322, 324, 326 measurements that estimate the volume of water extracted through the membrane 395 into the fluid sample pump 330. For the most reliable and repeatable results, at least one microfluidic gate (not shown) may be added to allow a fluid sample 16 to enter the device, then the gate could close to prevent, or adequately slow, introduction of new fluid into the channel 380. Integration of microfluidic gates will be further taught in later figures and embodiments. The aspect ratios of the channel 380 shown in FIG. 3 are for diagrammatic purposes only, and the channel 380 could be very long with a small cross section, e.g., a coiled tube.

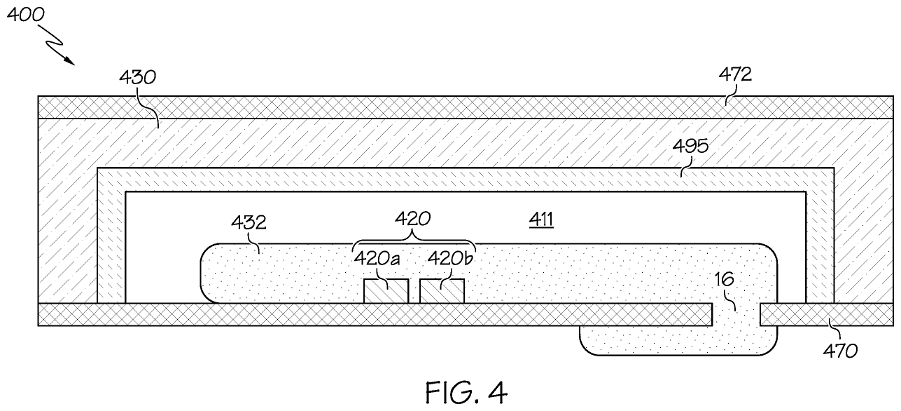

With reference to FIG. 4, where like numerals refer to like features of previous figures, a fluid sample coupler 432 wicks and holds a fluid sample 16. Alternately, fluid sample coupler 432 could be a channel, capillary, or wetted surface with fluid 16 at least partially exposed and unconfined (allowing evaporation). Outside the fluid sample coupler 432 is an air or gas gap 411, followed by a water vapor porous concentrator or membrane material 495, followed by a fluid sample pump 430 comprised of, e.g., a desiccant. The air gap 411 is used, because in some cases, small ions or other target analytes will penetrate through an osmosis membrane as described in previous embodiments. Therefore, the air gap 411 will only allow exit of volatile compounds such as water, and retain more solutes than a membrane could, in some cases. An external film 472 that is not water vapor permeable is also provided, to prevent desiccant from becoming saturated due to moisture or water vapor from outside the device 400. In an alternate embodiment, the device may omit the pump and film, and will therefore operate by evaporation of the fluid sample into ambient air (not shown). Other beneficial features may be included (not shown) such as a heater to promote evaporation, for example. The heater could be integrated onto substrate 470 between fluid sample coupler 432 and substrate 470, and could be, for example, a simple heater based on electrical resistance. To prevent the device wearer from feeling heat on their skin, low thermal conductivity or thermal isolation materials may also be added between the heater and skin (not shown). The disclosed invention may include at least one air gap as a pathway for evaporation concentration of fluid, and may include at least one desiccant that receives water evaporated from the fluid, and may include at least one heater that promotes evaporation of water from the fluid. Similarly, vacuum pressure could also be applied to promote evaporation.

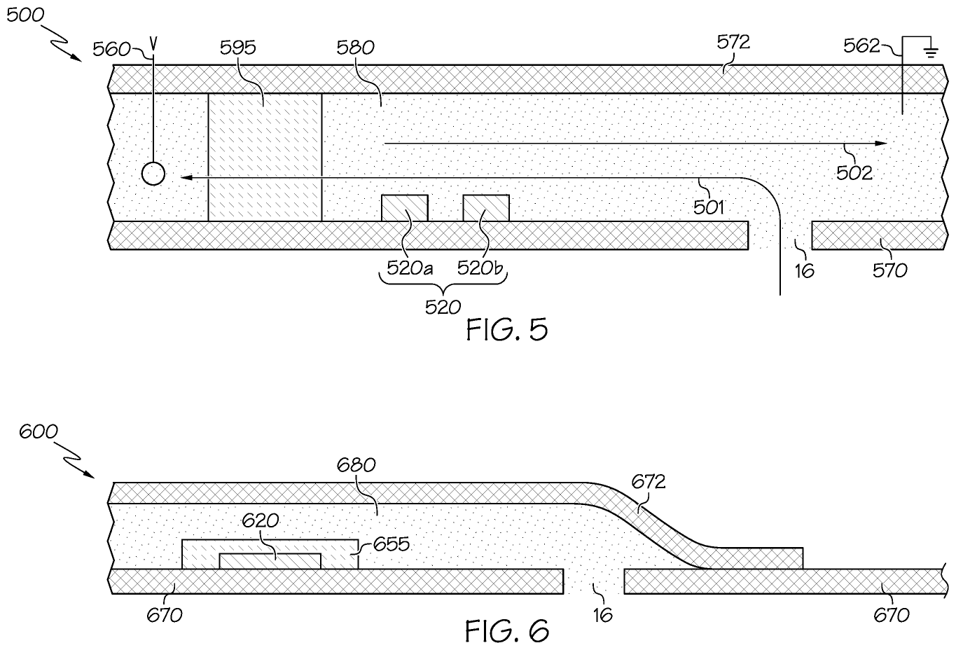

With reference to FIG. 5, where like numerals refer to like features of previous figures, a microfluidic channel 580 is formed between film or material 570 and film or material 572. Like previous embodiments, the concentrator membrane 595 is permi-selective in terms of which analytes may pass through it, or concentrator 595 could pass primarily only water. In such embodiments, sample concentration is achieved by one of several active microfluidic mechanisms, such as electrophoresis, iontophoresis, electro-osmosis, dielectrophoresis, electro-wetting, pressure-driven advective flow, etc. For example, as shown in FIG. 5, if the driving force were electrical, electrodes 560 and 562 could provide voltage or current, and therefore drive a flow of fluid sample 16 containing a target analyte leftward in the direction of arrow 501. Concentrator membrane 595 would block the target analyte, (e.g., glucose), causing an increased molar concentration of the target analyte. In some embodiments, the membrane may also be configured to block a second target analyte for calibration purposes, (e.g., testosterone). The molarities of the two target analytes would be measured through use of sensors 520a and 520b, respectively. Such a device 500 could be used to take multiple measurements of the target analytes, the performance improved by removing the fluid sample after sensing by reversing the voltage, or by using other flow driving means, so that the fluid sample 16 moves away in the direction of arrow 502. In some embodiments, the present disclosure may include at least one component capable of creating a reversible flow of fluid. In other embodiments, the invention may include at least one component for applying a non-equilibrium pressure (e.g., electrical, mechanical, etc.) to reverse the fluid sample 16 flow.

With reference to FIG. 6, where like numerals refer to like features of previous figures, a channel 680 is formed between film or material 670 and film or material 672. A sensor 620 is coated or surrounded by an immiscible material 655. Immiscible material 655 is non-dissolvable in the fluid sample 16. The immiscible material could be an ionic liquid, a hydrocarbon, a liquid crystal, a porous polymer, or any material which has a distribution coefficient with respect to water or other fluid which is greater than 2. For example, if immiscible material 655 were a fluid or gel that is more hydrophobic than water, then hydrophobic solutes in sweat or interstitial fluid, such as cortisol, lipids, or other solutes, would passively concentrate into immiscible material 655. The distribution coefficient is k and can be predetermined or measured, and the target analyte molarity that occurs will equal k, and therefore the target analyte sweat concentration can be easily predicted. Distribution coefficients can be in the 1's to 10's even 100's or more. An example would be electrochemical sensing of hemoglobin using glassy carbon surrounded by an ionic liquid. The analytes which have the greatest distribution coefficients with respect to sweat generally make electrochemical sensing more challenging, and therefore other methods of sensing, such as optical techniques may be preferred.

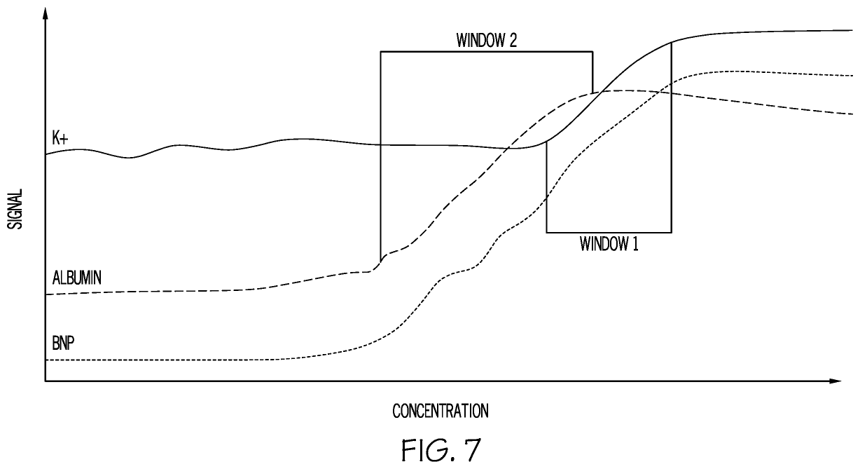

With reference to FIG. 7, a graph is provided to illustrate one example of how the invention could be utilized. At low concentrations of an analyte, there is no signal change (the concentration is below the limits of detection) and at high concentrations, sensor signals can become saturated. The fluid sensing device may measure continuously or repeatedly to determine whether a proper sensing window had been achieved. For example, because K+ maintains a fairly consistent sweat concentration relative to changes in sweat rate, K+ could be used to determine the molarity of a concentrated sweat sample. This molarity measurement would indicate when an adequate concentration of another analyte, for example the peptide BNP, had been reached to enable an accurate measurement of its concentration. The K+ measurement could then be used to back-calculate the BNP molarity in unconcentrated sweat. This is represented by window 1 in FIG. 7. Similarly, window 2 represents the range for which sweat concentrations of albumin would be used to determine the molarity increase of the sweat sample (because albumin concentration is fairly constant in blood). In one embodiment, an analyte-specific sensor may be continually operating to measure K+, albumin, or another marker with fairly consistent sweat concentration, and other analyte-specific sensors would be activated only when a target analyte reached an appropriate concentration window. As will be taught in later embodiments, devices can also adapt the amount of sample concentration to maintain the sweat sample in the sensing windows as exemplified in FIG. 7.

With reference to FIG. 8, where like numerals refer to like features of previous figures, an amperometric sensor 820 and enzymatic material 857 is provided, along with a secondary sensor 822 which can measure fluid collection rate or fluid generation rate. For example, sensor 822 could be a thermal flow sensor operable in the range of 0.1 nL/min to 100 nL/min. Amperometric sensors are a type of analyte-consuming sensor, which reduce the amount of target analyte present in the fluid sample by performing an enzymatic conversion of the analyte, allowing measurement to occur. Other sensor technologies that consume or irreversibly alter the target analyte may also be used, especially those where sample volume can limit proper sensor function. Such sensor modalities are useful for measuring analytes like ethanol, glucose, or lactate. Assume, for example, that sensor 820 and material 857 are configured to facilitate the amperometric sensing of ethanol. During the measurement process, the analyte undergoes a two or more step process of enzymatic conversion, followed by a charge transfer to or from sensor 820. Since analyte-consuming sensors deplete the available analyte, if they are continuously operated, the steady-state detection signal may remain below the sensor's lower limit of detection, or below the level of background electrical noise.

An embodiment of the disclosed invention allows continuous sensing with analyte-consuming sensors by periodically sampling only when a chronologically assured new (or unmeasured) fluid sample is introduced to the sensor 820, and after a sufficient amount of analyte is enzymatically converted. The flow sensor 822 measures the rate at which new fluid enters the device 800, which allows the device to determine when the fluid sample is fully refreshed. Once the chronologically assured new fluid sample is introduced to sensor 820, and after at least some of the target analyte is enzymatically converted, the device activates sensor 820 to sense amperometric charge. As a result, instead of continuous measurement, the sensor 820 only operates periodically, which allows the analyte concentration to build during intervals between measurements, which increases the signal relative to the lower limit of detection, or relative to the noise level. In another embodiment, flow sensor 822 is absent, and sensor 820 may be activated periodically, or according to a predetermined schedule. This example embodiment merely illustrates one device configuration that improves the function of enzymatic and other analyte-consuming sensors when used with sample concentration.

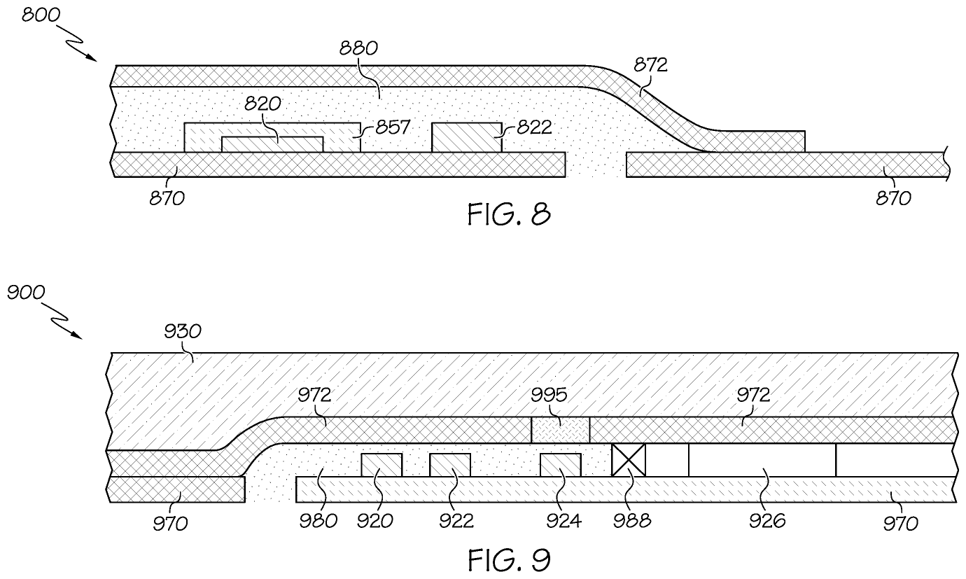

With reference to FIG. 9, where like numerals refer to like features of previous figures, a device 900 includes a plurality of different sensor types that may be used at different times. The device depicted in FIG. 9 is an illustrative example, but the invention is not so limited (multiple analytes may be sensed using various device, material, and sensor configurations). Sensor 920 is for estrogen, and sensor 922 is for progesterone, both of which have electrochemical aptamer-based sensors configured to operate at the analytes' natural concentration ranges found in biofluid. In the appropriate concentrations, these analytes can indicate the likelihood of impending female ovulation. Concentrator membrane 995 is permeable to solutes with size <1000 Daltons (Da), and relatively impermeable to solutes with size >1000 Da, such that estrogen or progesterone are able to pass through the membrane 995 (generally, size selective). Sensor 924 is any sensor type that measures one of an analyte, flow, or property of biofluid, which can be used to indicate the fluid sample's increase in molarity for analytes >1000 Da. Note, the channel 980 near substrate or material 970 and membrane 995 could be larger than shown, and therefore, this, like other embodiments, should not be strictly interpreted by the apparent dimensions in the figures. The device 900 is applied, for example, at 8 PM, and the next day, the user has selected 8 PM as a moment to determine the likelihood of ovulation within the next several hours. During device operation, sensors 920 and 922 measure the likelihood of ovulation by measuring estrogen and progesterone concentrations. Sensors 920 and 922 are able to measure estrogen and progesterone molarities in an unconcentrated biofluid sample, because these analytes are ˜300 Da in size, and will pass through membrane 995 into fluid sample pump 930. As biofluid continues to enter the channel 980, larger solutes unable to pass through the concentrator 995 will become concentrated in the biofluid sample. At a size of ˜30,000 Da luteinizing hormone will be unable to pass through the membrane 995, and with therefore be one of the solutes concentrated. At around 8 PM, a microfluidic gate 988, which could be any gate type known to those skilled in the art of microfluidics, allows the concentrated biofluid sample to flow onto sensor 926, which could be any type of sensor for luteinizing hormone, for example a lateral flow assay such as are commonly used in commercial urinary test strips for ovulation. If sensor 926 is a lateral flow assay, larger sample volumes may be required. Luteinizing hormone, like other proteins, is likely dilute in sweat compared to blood, but a device that collected sweat for 24 hours could collect and concentrate a sweat sample with a sufficient number of luteinizing hormone molecules to be detected by sensor 926. Sensor 924 may be used to inform the amount of concentration that has occurred, but would not be necessary if a simple qualitative measurement of the analyte is required. After stabilization, sensor 926 would then inform the user of the likelihood of ovulation.

This is an example of a device of the present disclosure that may be configured a number of different ways, and may include at least one microfluidic gate between a first sensor and the fluid sample that is being concentrated, an electrochemical sensor or a non-electrochemical sensor, a sensor for concentrated samples or a sensor for non-concentrated samples, or a sensor that does not receive a sample of fluid until one of the following occurs: 1) another sensor provides an input; 2) a scheduled time; or 3) a user provides an input or request. For example, if concentration of estrogen or progesterone were to change significantly in biofluid then signals from those sensors could go to electronics (not shown) which would then further trigger gate 988 to open or close as needed.

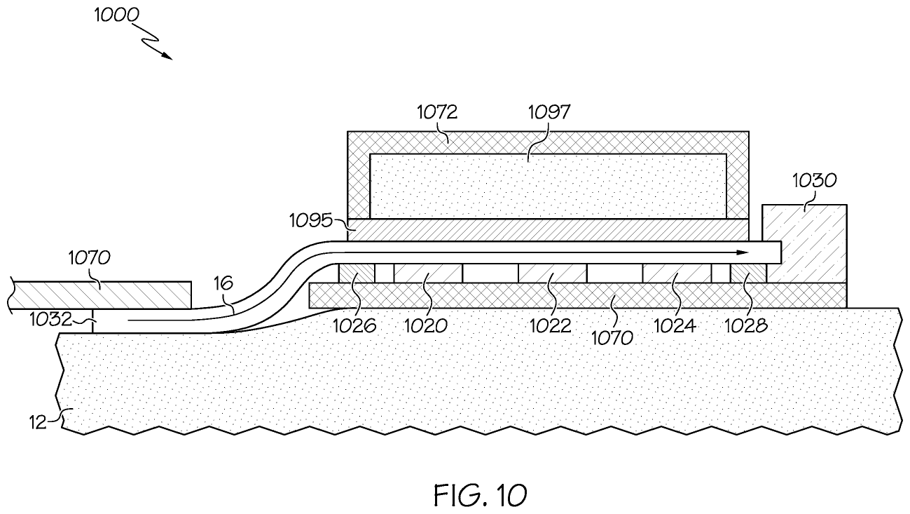

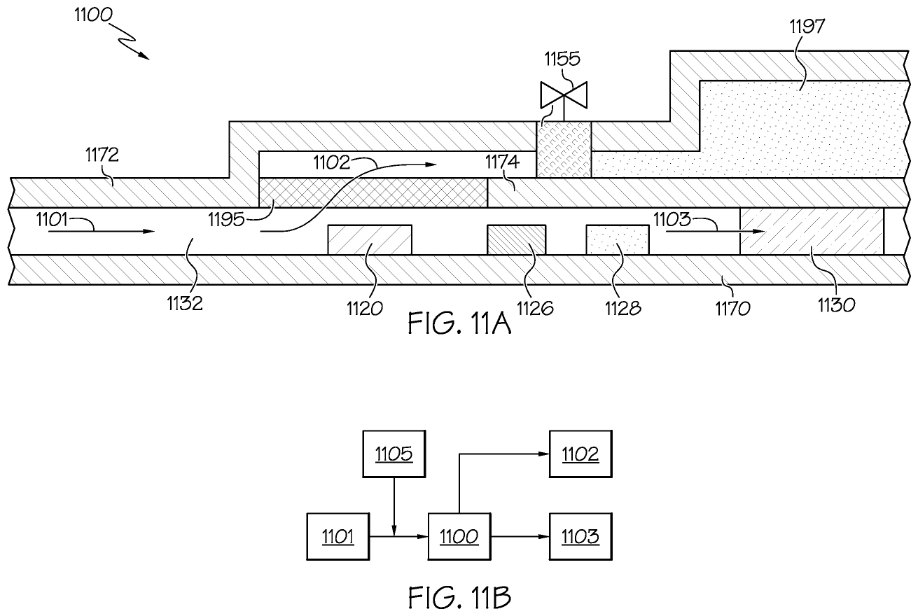

With reference to FIG. 10, where like numerals refer to like features of previous figures, a device 1000 includes a concentrator membrane 1095, such as a forward osmosis membrane, and a concentrator pump 1097. The concentrator pump 1097 could be comprised of a draw solution or material, like sucrose dissolved in water, or a dry draw material, e.g., a wicking material, hydrogel, dissolvable polymer, a large-molecule salt, dry sucrose, or other suitable materials capable of exerting a wicking or osmotic force or pressure. The device further includes a sweat collector 1032, which could be a cellulose film or a network of hydrophilic microchannels; fluid impermeable material or films 1070, 1072; a fluid sample pump 1030; fluid flow rate sensors 1026, 1028; and fluid analyte sensors 1020, 1022, 1024. As sweat is moved 16 along sweat collector 1032, water and certain sweat-abundant solutes will pass through the concentrator membrane 1095 and into the concentrator pump 1097, while the remaining sweat sample flows toward the fluid sample pump 1030. The sweat sample will accordingly become more concentrated with respect to the target analyte as it moves in the direction of the arrow 16 along the sweat collector toward the fluid sample pump 1030. The flow rate sensors 1026, 1028 could be mass thermal flow sensors, or another suitable sensor type. As the sweat sample is concentrated by the concentrator membrane 1095, the geometry of the sweat collector 1032 and the ratio of fluid flow at flow sensors 1026 and 1028 could be used to determine the total amount of fluid concentration achieved by the device. The disclosed invention may include at least one flow sensor or a plurality of flow sensors for determining the degree of concentration. The sensors 1020, 1022, and 1024 could be for the same analytes, or different analytes, or could be different sensor modalities, for example, they could all be configured to sense for cortisol. Sweat cortisol concentration would be sufficient to allow measurement as long as at least one of the sensors 1020, 1022, 1024 experienced the necessary concentration range for an accurate cortisol reading. Therefore, the disclosed invention may include a plurality of sensors for the same analyte, wherein at least one of said sensors measures a fluid sample that is more concentrated with respect to a target analyte than the fluid sample measured by at least one other of said sensors.

With further reference to FIG. 10, in an alternate embodiment of the disclosed invention, each of sensors 1020, 1022, 1024 could further contain two subsensors, one subsensor may be an electrochemical aptamer-based sensor for albumin, and the other subsensor may be an electrochemical aptamer-based sensor for luteinizing hormone. Because an individual's blood albumin concentration is usually constant, albumin could serve as a reference analyte for a target analyte that does show significant blood concentration variation (e.g., luteinizing hormone). Such an arrangement and use of two sensors can help increase the analytical accuracy of the device, especially since albumin and luteinizing hormone are large, and most types of filtration membranes that can be used in the disclosed invention would be impervious to their passage. Therefore, the invention may include at least one sensor specific to a reference analyte, where said reference analyte is concentrated to a similar degree as a target analyte, at least one sensor specific to the target analyte, and where concentrations of the reference and target analytes can be compared.

In another alternate embodiment, a first sensor can measure the fluid concentration of a reference analyte (e.g., albumin) before sample concentration, and a second sensor can measure the reference analyte concentration after or during sample concentration. Sample concentration as disclosed complicates analyte sensing, because most sensing modalities have a limited dynamic range (e.g., EAB sensors typically have a dynamic range of between −40× to +40× the aptamer's linear range K_{D}), which means that sample concentration (e.g., 10× or more) and biological concentration variances (e.g., 10× or more) can put analyte concentrations outside the dynamic range of the sensors. Therefore, sensors may be arranged along the sweat collector 1032 so that their dynamic ranges increase as sweat moves in the direction of the arrow 16. For example, sensor 1020 and its subsensors for albumin and luteinizing hormone could have a dynamic range centered at lower concentrations than the dynamic range for sensor 1022 and its subsensors for albumin and luteinizing hormone, and 1024 could have dynamic ranges centered at the highest concentrations. Embodiments of the disclosed invention may, therefore, include a first sensor for measuring a fluid analyte concentration, and a second sensor for the fluid analyte concentration, where the second sensor has a dynamic range of detection that is centered on a higher concentration (K_{D}) than that of the first sensor.

With further reference to FIG. 10, some materials comprising the concentrator membrane 1095 need to be stored in a primarily wet condition, and some membrane materials need to be stored in a primarily dry condition. For dry storage materials, concentrator pump material 1097, such as a draw solution, can be introduced near or at the time of first use by numerous methods, including injection by a syringe, or use of foil burst valves, like those used in other types of point-of-care diagnostic cartridges.

With further reference to FIG. 10, in an alternate embodiment, the degree to which a target analyte will be concentrated by the device while in use is easily predictable. Generally, achieving analytical accuracy becomes more challenging as fluid generation rates or fluid sampling intervals change, because the amount of concentration produced by the concentrator pump disclosed herein varies with variations in fluid flow rates through the device. The flow rate of, e.g., sweat through the device depends on the inlet flow from skin, the outlet to the fluid sample pump 1030, and a flow of at least water into the concentrator pump 1097. To reduce this variability, in some embodiments, the device is configured so that the degree of sample concentration for a target analyte is predetermined or predictable based on the specific ion concentrations or the total ionic strength/osmolarity in the sample fluid or in the concentrator pump 1097. For example, the concentrator membrane 1095 could be a membrane that allows mainly water transport, but is impervious to the target analyte, lactate. Sensor 1026 could measure a fluid lactate concentration before the sample is concentrated. Next, assume that the incoming sweat flows into the device at a sweat generation rate that produces ˜0.20 mM concentration of lactate in the concentrated sample. Concentrator pump 1097 would be configured with a draw solution containing 400 mM in lactate concentration and have other solutes that match natural sweat concentrations or that match the general (total equivalent) osmolality of sweat, except for the additional osmolality contribution of the 400 mM lactate concentration. The concentrator membrane would be long enough or large enough (e.g., mm's or cm's long) so that the fluid sample in sweat collector 1032 loses water until it also reaches 400 mM lactate concentration by water loss, resulting in ˜20× concentration. Importantly, this degree of concentration could be accurately determined prior to device use because a sensor 1020 measures the unconcentrated fluid lactate concentration and the concentrator pump 1027 has a draw solution with a known lactate concentration (purposely configured). Alternately, the target analyte need not be measured in unconcentrated fluid if the analyte concentration varies little in the fluid, or if an application does not require a high degree of analytical accuracy.

In an alternate embodiment, a device's target analyte concentration can be predetermined or predicted where the device measures the ionic strength or conductivity in the sample fluid and uses a draw solution with a near constant osmotic pressure greater than that of the fluid (at least 2×). Maximum analytical accuracy will therefore be achieved if sensors 1022, 1024 for target analytes are near the end of the concentrator membrane 1095 (near the fluid sample pump 1030), where lactate (or ionic strength) in the fluid sample would be near or equal to the concentration of lactate (or other draw solution) in the concentrator pump 1097. Lactate is not the only possible example, since Na+ and Cl− are also possible targets, especially if draw materials utilize materials such as MgCl₂ or CeCl₃ which will have greater difficulty leaking back into the fluid sample from the concentrator pump 1097 (divalent cations, etc.). Alternatively, uncharged solutes can be used, including sugars. Finally, polyelectrolytes, both positively and negatively charged, can be used as additional draw solutions including but not limited to polyacrylic acids, polysulfonic acids, polyimidazoles, polyethyleneimines, etc. The disclosed invention may therefore provide a determined amount of sample concentration, where at least one first solute in the concentrator pump is also a solute in the fluid, and the concentration of the first solute in the concentrator pump is greater than that in the fluid by at least 2× to enable sample concentration by osmosis. The invention may also include at least one sensor to measure the first solute's concentration in an unconcentrated fluid sample.

With reference to FIG. 11, where like numerals refer to like features of previous figures, a device 1100 includes an incoming flow path 1101 and two exit flow paths 1102, and 1103. A microfluidic or other type of controllable valve 1155, such as a PDMS pneumatic control valve, is provided to control fluid flow to concentrator pump 1197. In one example embodiment, the draw rate of concentrator pump 1197 would be sufficient to reduce fluid flow through path 1103 to zero if valve 1155 were fully opened. The device therefore works as follows: sensors 1126 and 1128 detect the presence of fluid, and are used to provide feedback control for valve 1155. Valve 1155 would be configured to control fluid flow so that sensor 1126 is wetted by fluid, but sensor 1128 remains unwetted. As a result, the device would ensure that at least one analyte-specific sensor 1120 is wetted by the fluid sample being concentrated. Using one or more techniques described herein, once the target analyte is sufficiently concentrated to allow the sensor 1120 to take an accurate reading, the valve 1155 may be partially or completely closed, restricting flow path 1102. Restricting flow path 1102 activates flow path 1103, and the (old) fluid sample moves away from sensor 1120 and onto fluid sample pump 1130. In this configuration, the device could repeatedly concentrate a fluid sample, sense the analyte, and then eliminate the fluid sample in preparation for another sensing event. Therefore, embodiments of the invention may include at least one tunable valve that controls the amount of sample concentration that occurs.

With reference to alternate embodiments, components taught for FIG. 11 may be extended to more general embodiments such as that illustrated in FIG. 11B. Component 1101 introduces the fluid to be measured, component 1105 introduces an unconcentrated fluid such as water, saline, buffer, or other fluid, component 1100 is where sample concentration may occur and where the amount of water loss due to concentration is regulated by component 1102, and component 1103 is a pump like that taught for previous figures. Therefore, the invention may include a plurality of valves, or an inlet valve for at least one unconcentrated fluid. In some embodiments, the sample concentration component could become clogged as a high concentration of solutes in the tested fluid buildup. Therefore, component 1105 could introduce an unconcentrated fluid such as water, which could be used to flush the device and clear the highly concentrated solutes. FIG. 11B also generally teaches that valves and inlets could be placed at multiple locations. For example, component 1101 could have a valve that regulates the introduction of fluid sample into the device. Therefore, the disclosed invention may include at least one valve that controls the flow rate of a fluid that is unconcentrated.

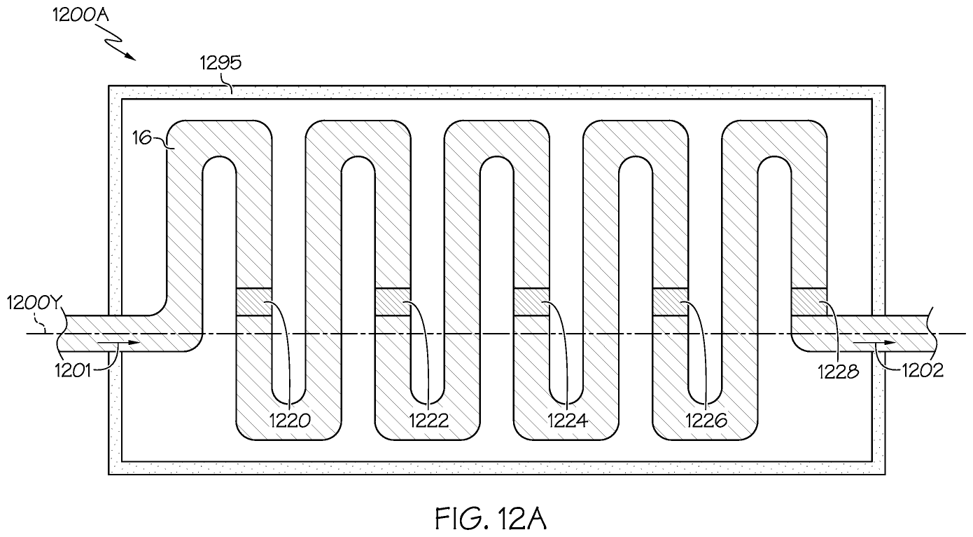

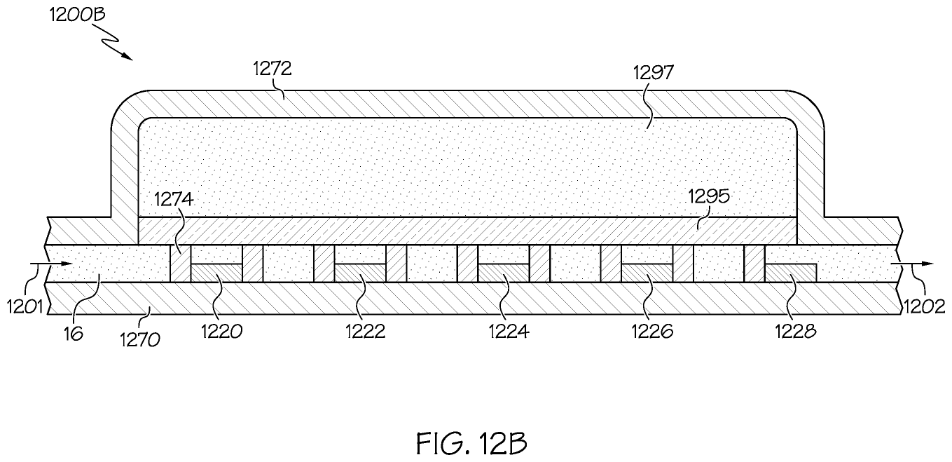

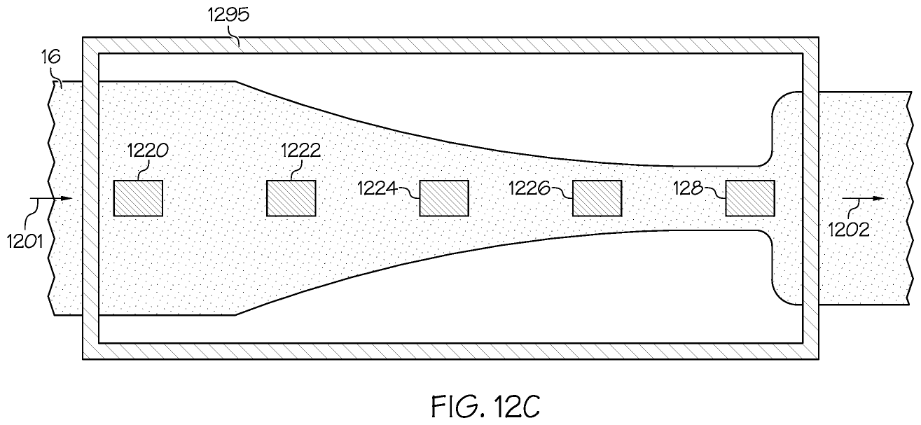

An example embodiment of the device described in FIG. 11 requires several controls and sensors that may be unnecessarily complex or sophisticated for some applications. With reference to FIGS. 12A and 12B, where like numerals refer to like features of previous figures, a simpler device 1200 includes a top view diagram 1200A and a side view diagram 1200B, which depicts a cross section of 1200A along axis 1200Y. A fluid sample 16 enters the device at opening 1201 and flows inside a channel that is constrained on its upper surface by a concentrator membrane 1295 or fluid impermeable material 1272, and ends in opening 1202. A plurality of sensors 1220, 1222, 1224, 1226, 1228 are provided within the channel. As the fluid sample 16 moves along the channel, a concentrator pump 1297 causes water (and in some embodiments certain small fluid-abundant solutes) to pass through the concentrator 1295. If the fluid flow rates are very low, then only sensors nearer to opening 1201, such as sensors 1220 and 1222, may experience analyte concentrations sufficient to allow accurate measurements. Sensors farther along the channel, such as 1226 and 1228, will remain unused if the fluid 16 does not reach them, or their data discarded if the analyte concentration remains inadequate. Conversely, at very high fluid flow rates, sensor 1228 may be the only sensor to receive sufficiently concentrated analytes. In some embodiments, opening 1202 could be configured adjacent to a fluid sample pump (not shown). Or concentrator membrane 1295 and concentrator pump 1297 could both concentrate the fluid sample and supply wicking pressure to move fluid 16 through the channel. A specific example may be taught through FIGS. 12A and 12B. Assume 2 nL/min/gland sweat generation rate, 10 eccrine sweat glands under the device, and sweat collected from 0.1 cm₂ area. This would provide a sweat flow rate of 20 nL/min, or 100 nL every 5 minutes. Assume 10,000× concentration of the fluid sample by the end of the channel (0.01 nL) and by the end of 5 minutes. Assume the draw rate of the forward osmosis concentrator membrane is 200 nL/min/mm₂ or 1000 nL/mm₂ every 5 minutes. Assume a channel that is 500 μm wide by 50 μm high, with additional spacers added to the middle of the channel if needed for support of the channel height. If the channel is 2 cm long, then it has a volume of 2·(500E-4)(50E-4)=5E-4 mL or 500 nL. This channel would tolerate a fluid flow rate up to a maximum of 10 nL/min/gland, which is unlikely to be encountered, meaning the channel as disclosed would be able to accommodate all typical sweat generation rates.

With further reference to FIG. 12, consider a case where the sensors are electrochemical aptamer-based sensors with an attached redox couple. Such sensors typically have a linear range of 80× the K_{D} value for the target analyte. If 100× sample concentration were needed, as little as 2 to 3 sensors could achieve a proper reading within range. The distance between the sensors is known, and therefore the amount of concentration measured from sensor to sensor could be used to determine the flow rate through the channel (the concentrator membrane flow rate out of the channel would also be known). This could then be used to back-calculate the original analyte concentration in the fluid sample. Furthermore, flow rate could be determined by simply knowing which sensors are wet with a sample of fluid, which would then allow calculation of the flow rate of fluid coming into the concentration portion of the device. Therefore, the disclosed invention may include a plurality of sensors that determine a fluid flow rate into the device by measuring an unconcentrated analyte concentration and comparing it to a concentrated analyte concentration. Furthermore, if the length of the channel is known, and the concentration difference between successive sensors measured (e.g., between 1222 and 1224), then the device may determine the concentration increase per unit length of channel, and thereby determine the total amount of concentration increase at each sensor.

With reference to FIG. 12C, in an alternate embodiment, the channel containing fluid 16 can be tapered or geometrically reduced in any manner that minimizes the reduction in fluid flow velocity caused by volume loss as the fluid moves through the sample concentration component of the device and water is extracted by the concentrator membrane 1295. If a device achieved a high degree of fluid sample concentration, there could be very little fluid sample volume (and hence little fluid flow) left by the time fluid exits the concentration component. Therefore, to ensure an adequate flow of fluid through the device, the channel dimensions reduce along the channel in the direction of fluid flow.

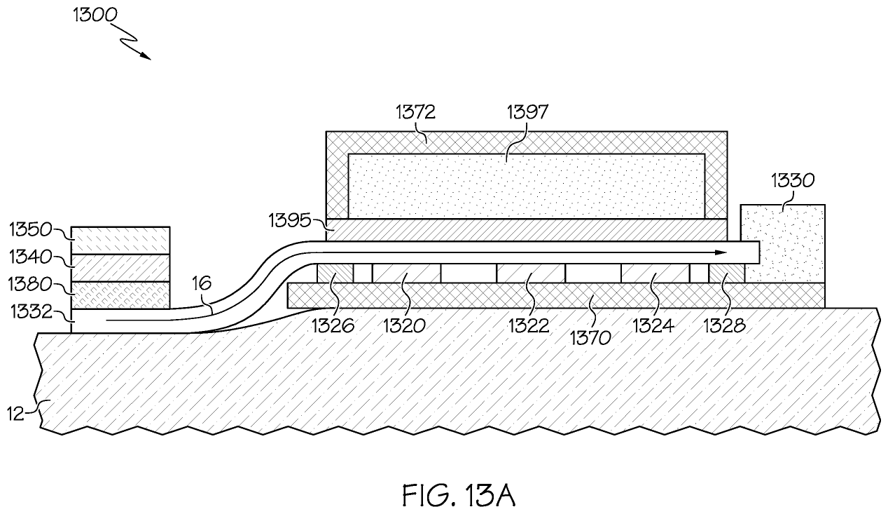

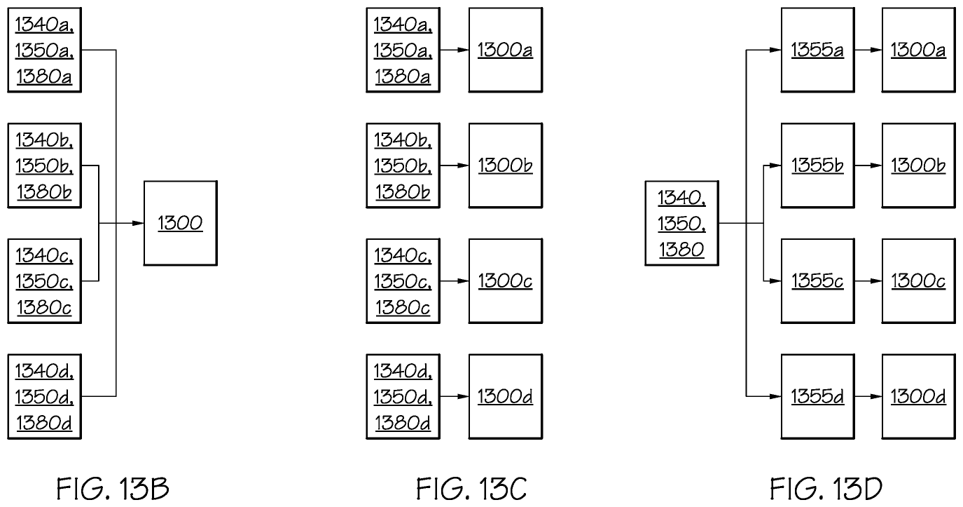

With reference to FIG. 13A, which is a variant of the previously taught FIG. 10, a device 1300 may include sweat stimulation and/or reverse iontophoresis or iontophoresis capabilities. For example, component 1350 could be an electrode, and component 1340 is an agar gel with pilocarpine or carbachol, and component 1380 is a track-etch membrane or other suitable membrane to reduce passive diffusion between the wicking component 1332 and the gel 1340. As a result, the device 1300 is capable of integrated sweat stimulation. Alternately, component 1340 could be a gel containing a buffer against changes in potential of hydrogen (pH), and electrode 1350 used to extract analytes in part from the body by reverse iontophoresis in order to increase their concentrations in sweat.

With reference to FIG. 13B, the components taught for FIG. 13A could be arranged such that a plurality components for sweat stimulation or reverse iontophoresis (such as 1340a, 1350a, 1380a being one of such components) may feed into a common device 1300 that is capable of sample concentration. Such a design could prove useful, for example, where only a certain sweat flow rate into a device 1300 is needed and the number of sweat stimulation components utilized could be chosen to provide the most suitable total flow rate of sweat into the device 1300.

With further reference to FIG. 13C, multiple such components could also be used individually where sweat stimulation or reverse iontophoresis components could feed into sub-devices with their own sensors (e.g., a 1300a). Such an embodiment would be particularly useful where sub-devices such as 1300a, 1300b, 1300c, etc., are lateral flow assays or other sensor modalities that can be utilized only once.

With reference to FIG. 13D, in yet another alternate embodiment, multiple valve components (1355a, 1355b, 1355c etc.) could be used to control, initiate, or stop flow of sweat to one or more sub-devices 1300a, 1300b, 1300c, etc. In this example, sweat comes from a single common component 1340, 1350, 1380, but could also use multiple sources as taught for FIGS. 13B and 13C.

In one embodiment, a functionalized silica gel, silicon dioxide nanoparticles, or other suitable substrate, can be added to a concentrator channel surface so that the surface has a high affinity for a target analyte through physi-sorption or chemi-sorption. Such a functionalized surface becomes the stationary phase of the concentrator channel. When fluid, as the mobile phase, is introduced into the device and flows past the surface, the target analyte is retained on the surface while the fluid continues to flow. The surface may be forced to release the target analyte by changing the fluid composition, e.g., by adding a solvent, changing the pH, changing solute concentrations, changing temperature, introducing electromagnetic radiation, or other system parameter. If the substrate is in the proper form, such as a bead or nanoparticle, multiple configurations may be present within the same concentrator/retarder system. This will allow the system to simultaneously concentrate multiple analytes using a single channel or using at least fewer channels than target analytes. The device as disclosed can be used to increase the concentration of analytes of interest, functioning similarly to the way a chromatography column is used for purification.

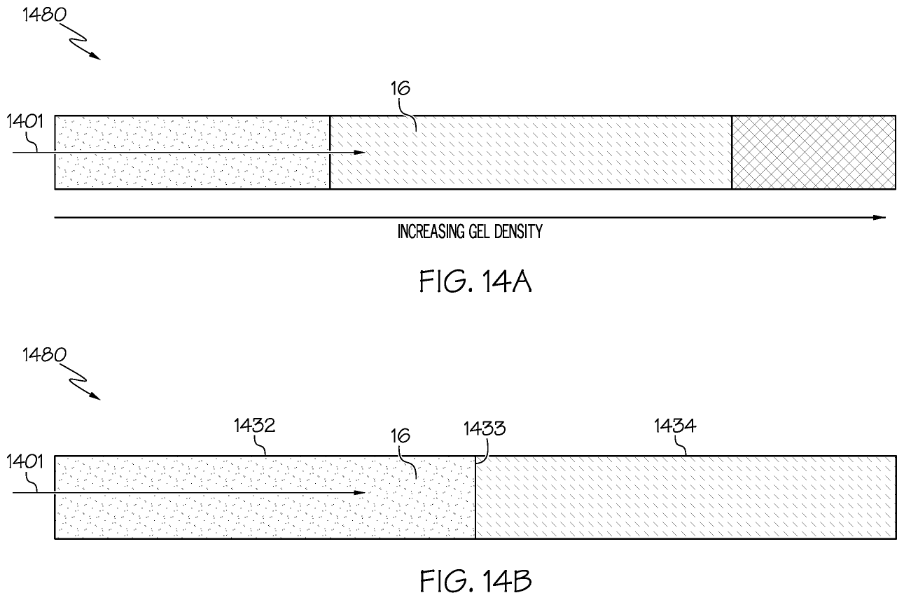

With reference to FIG. 14A, another embodiment of the disclosed invention would use a channel 1480 that includes a gel (or other medium) that possesses a gradient in density or pore size in the direction of the fluid flow 1401. The pore size may be tuned to correlate with the size of one or more target analyte(s). As the fluid flows through the gel, the analytes that are larger than the pores will move slower than the flow rate. As the pore size decreases, the analyte flow rate will therefore decrease proportionally. As the analyte flow rate slows relative to the fluid flow rate, the analyte will gradually become concentrated in the direction of flow 1401. A similar embodiment depicted in FIG. 14B would configure two or more gels (or other media) with different densities in the channel 1480. As depicted, a first section 1432 has a first density, and a second section 1434 has a second, greater density. Step edges of increasing densities are thereby created at the boundary 1433 between the sections. These step edges will cause the analyte to concentrate at the boundaries and move at a slower rate in the next section. The result is a “wave front” in the channel in which the target analyte exists at a higher concentration than it occurs in unconcentrated fluid. Sensors could be placed within these sections to characterize the analyte concentration factor to facilitate converting the concentrated value back to the unconcentrated value.

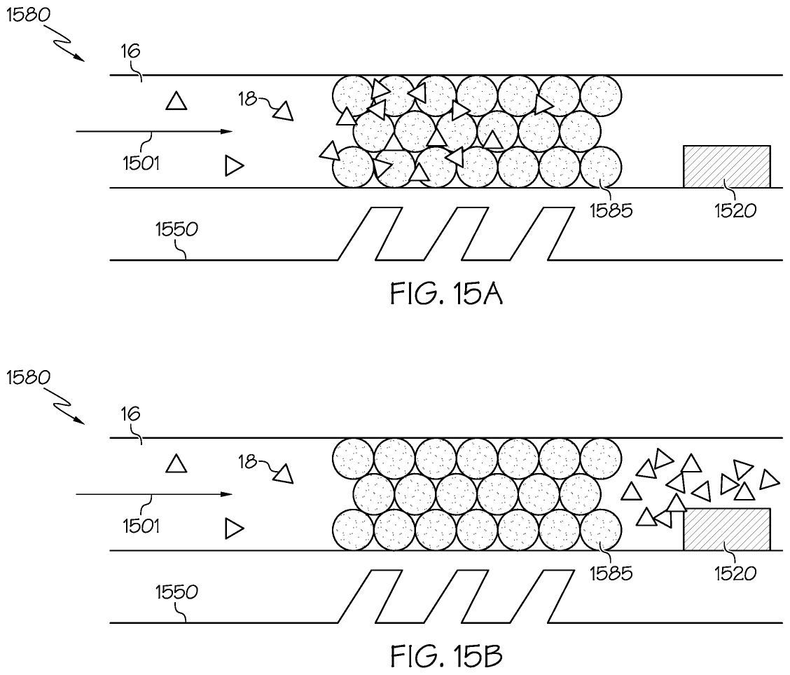

With reference to FIG. 15A, another embodiment illustrating fluid sample concentration includes a channel 1580 with a plurality of microfluidic capture beads 1585. As a fluid sample 16 flows through the channel 1580, molecules of the target analyte 18 are trapped by the capture beads 1585. The embodiment further includes an analyte-specific sensor 1520 for measuring the target analyte, well as a heating element 1550. With reference to FIG. 15B, the fluid sensing device activates the heating element 1550, causing the target analyte molecules 18 to dislodge from the beads 1585 and flow with the fluid sample across the sensor 1520.

There are many applications where samples must be concentrated before analysis, including, without limitation, biofluids, fuels, wastewater, municipal water, environmental fluid sources, as well as food safety and/or quality applications. The embodiments of the disclosed invention apply broadly to these other fluid and analyte systems, and other point-of-use scenarios, so long as they rely on similar mechanisms for integrated sample concentration and analyte sensing. Not all embodiments will be taught in this way, rather it will be apparent from the additional specification below how all embodiments may cover more broadly other fluids, analytes, and point-of-use scenarios with minimal modification.

With further reference to FIG. 3B, for example, the fluid sample 16 may be a liquid food sample of a variety of viscosities, including without limitation, condiments, beverages, juices, sodas, and mixes. Sensors 320, 322, 324, 326 may be configured to measure one or more analytes relevant to food safety and/or quality.

With further reference to FIG. 7, an embodiment of the disclosed invention is configured to collect and measure analytes in non-sweat biofluids, such as saliva or interstitial fluid. At low concentrations of an analyte, there may be no signal change (the concentration is below the limits of detection) and at high concentrations, sensor signals can become saturated. The fluid sensing device may measure continuously or repeatedly to determine whether relatively linear windows (Windows 1 and 2) are achieved. As for the example of K+ in sweat, a similar strategy of evaluating the degree of biofluid concentration by examining the concentration of a reference analyte that remains stable under most physiological conditions may be employed. For example, albumin concentration in blood remains relatively constant under most physiological conditions, and consequently, albumin concentration remains stable in biofluids that are blood filtrates (e.g., sweat, saliva, interstitial fluid). Therefore, the increase in albumin concentration may be used as a measure of the extent of concentration for such biofluids. Similar reference analytes exist for other fluids that will allow quantitative assessments of the degree of fluid concentration relevant to target analytes.

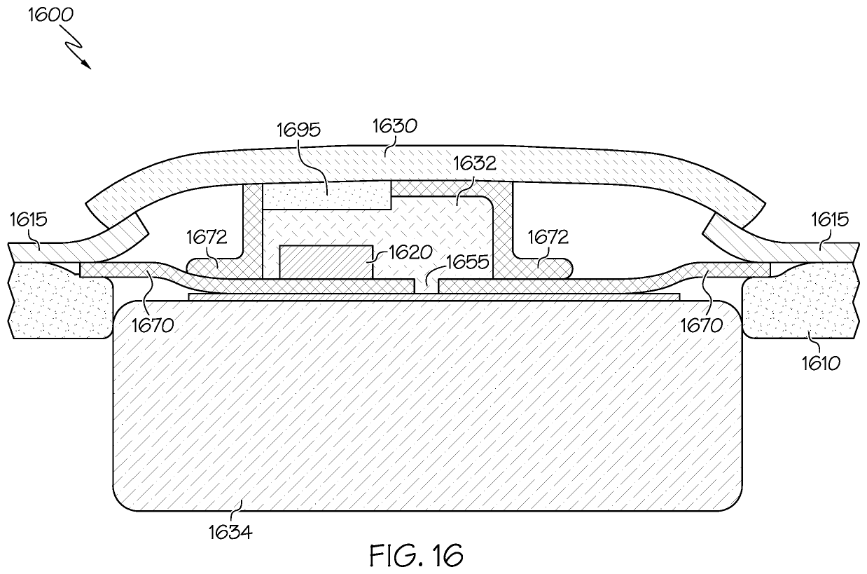

With reference to FIG. 16, where like numerals refer to like features and functions for FIG. 2, a device 1600 provides a wicking material, e.g., a sponge, 1634 configured to collect a sample fluid, such as river water, by placing the sponge into the water (not shown). The water would flow into the fluid sample coupler 1632 and across the sensor 1620. Water in the sample is drawn into pump 1630 through sample concentrator 1695 and is concentrated with respect to one or more target analytes, such as Cryptosporidium or one of that organism's products or toxins. The sensor 1620 then measures the analyte's concentration, or detects the analyte's presence, as the fluid sample concentrates.

With further reference to FIG. 6, the device 600 is configured to detect microbes in a fuel sample. For example, modern biodiesel is especially hygroscopic. The presence of water encourages microbial growth, which either occurs at the interface between the oil and water, or on storage tank walls, depending on whether the microbes are aerobic or anaerobic. In this case, the device 600 is placed in contact with the fuel within the tank, or a flow of fuel (e.g., within a hose transporting the fuel). Channel 680 would contain the fuel brought into the device, and the immiscible material 655 would be non-dissolvable in the fuel. In contrast to the disclosed device's use in sweat, the immiscible material 655 may be hydrophilic (e.g., a hydrogel with water), which would passively concentrate the target analyte by distribution coefficient compared to the fuel. The sensor 620 could then detect the target analyte, which may be a bacterium, a fungus, a virus, or a toxin or other product produced by those organisms.

With further reference to FIG. 10, the device 1000 is configured to detect a target analyte associated with a sexually transmitted infection, such as chlamydia or gonorrhea. The user would urinate onto wicking material 1032, and the device would concentrate and sense one or more target analytes indicative of the infection, such as an antigen, a product of the antigen, an antibody, a cytokine, or other analyte. Sensors 1026 and 1028 could be chloride sensors (e.g., bare Ag/AgCl electrodes, or ion-selective electrodes) whose ratios of potential determine the degree of concentration occurring as the urine sample is moved across the membrane 1095 toward the pump 1030. Sensors 1020, 1222, 1024 are configured to detect the disease analyte(s), and further, each sensor 1020, 1022, 1024 may be configured to detect the disease analyte at different concentrations. Such an arrangement would be useful for the described example, because the user would be able to have dilute urine or concentrated urine, and the device can accommodate this by operating over a wider range of these conditions for analyte concentration.

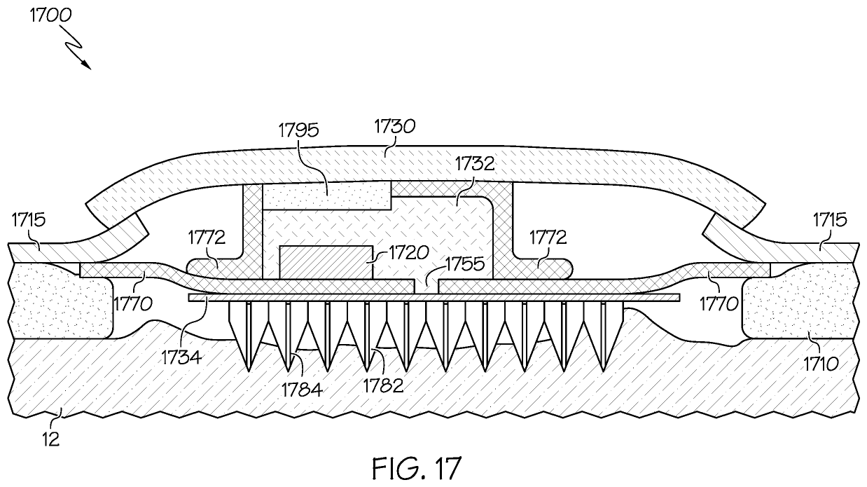

With reference to FIG. 17, a device 1700 is configured to extract a biofluid, such as interstitial fluid (ISF), from human skin in order to sense one or more analytes. Device 1700 includes a single microneedle or an array of two or more microneedles 1782 each having a distal tip adapted to penetrate the skin surface. The microneedles 1782 may be made of metal, plastic, ceramic, hydrogel, or other suitable material. The microneedles 1782 may include a hollow bore or lumen 1784 forming a flow path for conveying fluid into the device. The lumen may optionally be filled with a hydrogel (not shown) for wicking fluid through the needle, or in some embodiments, the microneedles are solid. Individual needles can range from 10's of μm to 100's of μm or mm's in length if pain is a non-issue. Lumens may range from 10's of μm to 100's of μm in diameter, as a non-limiting example. Microneedle arrays have a total area covering from 1 mm₂ to 3 cm₂ of skin surface. Optimal microneedle size and spacing allows complete penetration of individual needles into the epidermis layer of skin with minimal discomfort while maximizing extraction of interstitial fluid. The device 1700 includes a fluid impermeable substrate 1770 such as PET, and an adhesive layer 1710, which also functions to secure the device to the skin. Substrate 1770 has an opening 1755 to allow fluid to access one or more sensors 1720 (one is shown). Adhesives can be pressure sensitive, liquid, tacky hydrogels, which promote robust electrical, fluidic, and iontophoretic contact with skin. The device 1700 may further include fluid impermeable materials 1715 and 1772. Extraction or flow of interstitial fluid can be achieved by wicking pressures created by one or more materials 1734, 1732, 1730. Alternatively, the device 1700 is applied to skin, and then pressure is applied to the device toward the skin 12 surface, which puts the dermis under positive pressure, thereby causing interstitial fluid to move out of the dermis and into the device 1700.

The microneedle array 1782 has a side proximal to the device that is in fluidic communication with a biofluid collector 1734 and a fluid sample coupler 1732 to convey the interstitial fluid through the opening 1755 and across the sensor 1720. Alternatively, the microneedles 1782 can be in direct fluidic communication with the coupler 1732. Sensor 1720 could be any sensor configured to sense a particular analyte in interstitial fluid, such as an ion-selective electrode, enzymatic sensor, or electrochemical aptamer-based sensor. The coupler 1732 is in fluidic communication with a pump 1730, which is comprised of a textile, paper, polymer, or hydrogel, and that serves to maintain fluid flow through the device. Coupler 1732 could be a 5 μm thick layer of screen-printed nanocellulose with a weak binder and/or a hydrogel material to hold the cellulose together. The coupler 1732 should have greater capillary or wicking force than the pump 1730, which in turn should have greater capillary or wicking force than the microneedles 1782. In a preferred embodiment, the coupler 1732 would have the greatest capillary or wicking force relative to the other wicking materials, such as 1734 and 1730, so that the sensor 1720 remains wetted with biofluid.

With further reference to FIG. 17, the device 1700 also includes a sample concentrator 1795, similar in form to the sample concentrators described above, that comprises a membrane that is at least porous to water, but that is impermeable to the analyte that is to be concentrated. As interstitial fluid flows onto the pump 1730, by wicking through the concentrator membrane 1795, solutes are concentrated in fluid sample coupler 1732. The device may be configured to concentrate a target analyte in the fluid sample by at least 2× the unconcentrated molarity. Depending on the application, the target analyte may be concentrated at least 10×, 100×, or 1000× the unconcentrated molarity.

In an alternate embodiment, an osmosis membrane can be used as the sample concentrator 1795, where the membrane is water-permeable, but is impermeable to electrolytes, such as K+. The sensor 1720 may measure K+ and another sensor (not shown) may be configured measure a second analyte. Embodiments of the disclosed invention may accordingly be configured with a first sensor specific to a first analyte and a second sensor specific to a second analyte, wherein both the first and second analytes are concentrated. Similarly, additional sensors may be added to measure additional concentrated analytes.

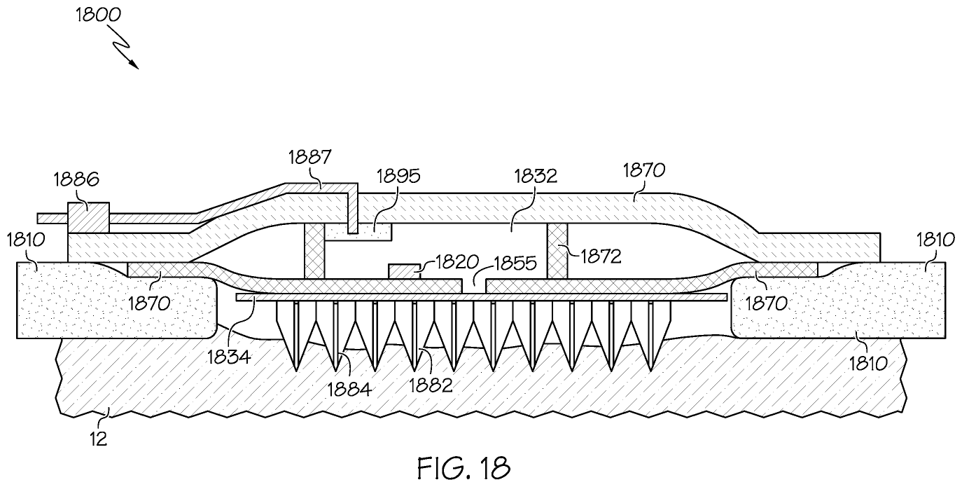

With reference to FIG. 18, wherein like numerals refer to like features of previous figures, a device 1800 can further comprise an active pump 1886 for drawing interstitial fluid through an array of hollow microneedles 1882 and into the device. Active pump 1886 can be, for example, a vacuum pump, a capillary force pump, a microdialysis pump, or a pulsatile vacuum pump. To facilitate fluid extraction by the active pump, a seal is formed between the device and skin around the microneedle array 1882 to prevent air entry from outside the device. As shown, the seal is accomplished by adhesive 1810 forming a seal with the skin 12 and with the fluid impermeable substrate 1870. For example, a top-down view of this embodiment would show the adhesive 1810 configured around and sealing off a central sampling area of skin where the microneedles are located. The active pump-assisted extraction can be used to pull interstitial fluid from the epidermis, through the lumens 1884 of microneedles 1882, and into a biofluid collector 1834.

With further reference to FIG. 18, the device 1800 also includes a sensor 1820, a channel 1832 surrounding the sensor, a concentrator membrane 1895, and a fluid impermeable material or films 1872. Pressure from pump 1886 draws sample fluid from collector 1834, through an opening 1855 in substrate 1870, and into sensor channel 1832. As the fluid sample is drawn into channel 1832, water and certain ISF-abundant solutes will pass through the concentrator membrane 1895 and exit the device through conduit 1887. The fluid sample surrounding the sensor 1820 will, accordingly, become more concentrated with respect to the target analyte as the fluid sample is drawn through the device.

In an alternate embodiment, the microneedles 1882 may be mounted on a microfluidic chip and attached to a syringe assembly through sterile tubing (not shown). The microfluidic chip can be used to secure the microneedles, and allows for an insertion depth of up to 2 mm into the skin surface. The syringe assembly can provide negative pressure to extract interstitial fluid through the hollow passageways in the microneedles and into a collection channel. From the collection channel, the fluid sample can be moved through the device using wicking or other pressure sources as described herein.

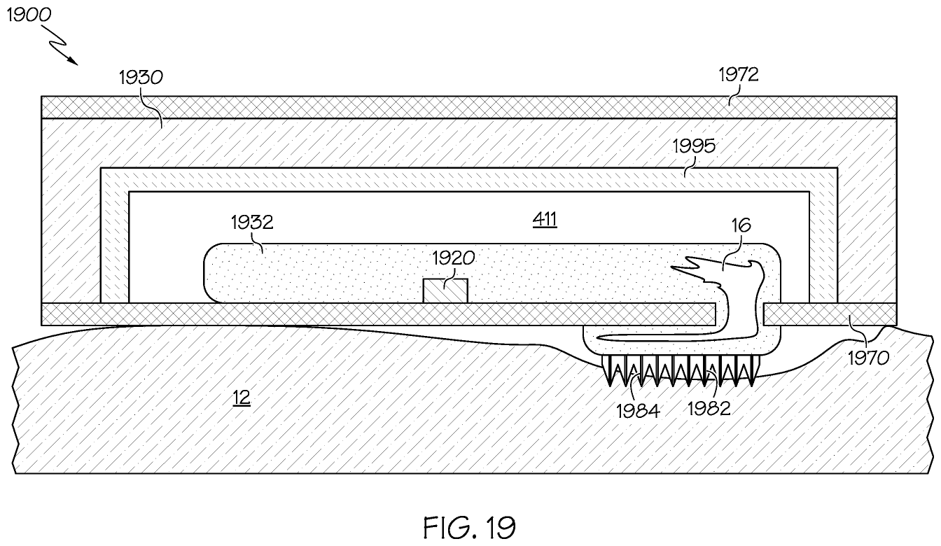

With reference to FIG. 19, which is a variant of the previously taught device of FIG. 4, a device 1900 includes a microneedle array 1982 for extracting an ISF sample from below the skin surface. As in the previous embodiments, each microneedle includes a distal tip for penetrating the skin 12 and a hollow bore or lumen 1984 for conveying interstitial fluid from the skin into the device. Microneedle array 1982 is fluidically connected to a coupler 1932 that wicks and holds a fluid sample 16. Alternatively, the coupler 1932 could be a channel, a set of capillaries, or a wetted surface, with the fluid sample 16 at least partially exposed to air and unconfined (allowing evaporation). Outside the coupler 1932 is an air or gas gap 411, followed by a water vapor porous concentrator membrane 1995, followed by a pump 1930 comprised of, e.g., a desiccant. Because small ions or other analytes can in some cases penetrate through an osmosis membrane, use of an air gap 411 will only allow exit of volatile compounds such as water, and retain more solutes than a membrane. An external film 1972 that is not water vapor permeable is also provided, to prevent desiccant from becoming saturated due to moisture or water vapor from outside the device. In an alternate embodiment, the device may omit the pump 1930 and film 1972, and will therefore operate by evaporation of the fluid sample into ambient air (not shown). Other beneficial features may be included such as a heater (not shown) to promote evaporation. The heater could be integrated onto substrate 1970, or located between the coupler 1932 and the substrate, and could be, for example, an electrical resistance heater. To prevent the device wearer from feeling heat on their skin, low thermal conductivity or thermal isolation materials may also be added between the heater and skin (not shown). The disclosed invention may include at least one air gap as a pathway for evaporation concentration of fluid, and may include at least one desiccant that receives water evaporated from the fluid, and may include at least one heater that promotes evaporation of water from the fluid. Similarly, vacuum pressure could also be applied to promote evaporation.

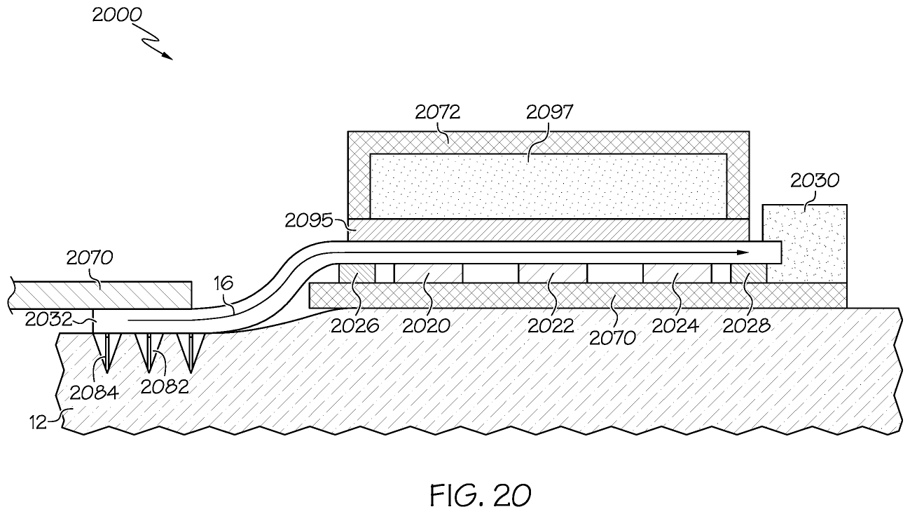

With reference to FIG. 20, which is a variant of the previously taught device of FIG. 10, a device 2000 for concentrating an interstitial fluid sample is depicted. The device includes a concentrator membrane 2095, such as a forward osmosis membrane, and a concentrator pump 2097. The concentrator pump 2097 could be comprised of a draw solution, like sucrose dissolved in water, or a draw material, e.g., a wicking material, hydrogel, dissolvable polymer, a large-molecule salt (sized to limit migration out of the membrane), dry sucrose, or other suitable materials capable of exerting a wicking or osmotic pressure on the ISF sample. The device further includes a fluid collector 2032, which could be a cellulose film or a network of hydrophilic microchannels. The fluid collector 2032 is in fluidic communication with the concentrator membrane 2095, a plurality of analyte sensors 2020, 2022, 2024 (three are shown), and sample pump 2030, and is configured to transport biofluid across and away from the sensors and to the sample pump.

The disclosed invention may include at least one secondary sensor 2026, 2028 (two are shown), which may be, e.g., a pH sensor, a flow sensor, or a plurality of flow sensors for determining the degree of concentration. In embodiments employing thermal flow rate sensors as secondary sensors 2026, 2028, the collector 2032 need only bring biofluid to adequate proximity with sensors 2026, 2028 to allow thermal exchange. Other secondary sensors may require fluidic communication with the biofluid sample. The device also includes fluid impermeable substrates or films 2070, 2072. Device 2000 additionally includes one or more microneedles 2082 attached to the fluid collector 2032. Microneedles 2082 each contain a lumen 2084, similar to those described above, for conveying interstitial fluid from the skin 12 to the fluid collector 2032.

As interstitial fluid is drawn through lumens 2084 and moved along fluid collector 2032, water and certain ISF-abundant solutes will pass through the concentrator membrane 2095 and into the concentrator pump 2097, while the remaining fluid sample flows toward the sample pump 2030. The interstitial fluid sample will accordingly become more concentrated with respect to the target analyte as it moves in the direction of the arrow 16 along the collector 2032 toward the pump 2030. As the fluid sample is concentrated by the concentrator membrane 2095, the geometry of the collector 2032, and the ratio of fluid flow at flow sensors 2026 and 2028, may be used to determine the total amount of fluid concentration achieved by the device. With interstitial fluid, osmolality is more constant than that of sweat, which can have wide variations in salinity or pH. Accordingly, when using osmotic preconcentration for an interstitial fluid sample, the amount of concentration of the sample can be more readily predicted without the need to measure the osmolality of the sample. The analyte sensors 2020, 2022, 2024 could be for the same analytes, or different analytes, or could be different sensor modalities, for example, they could all be configured to sense cortisol. The degree of sample concentration with respect to cortisol would bring cortisol concentrations to within the limits of detection of at least one of the sensors 2020, 2022, 2024. Therefore, the disclosed invention may include a plurality of sensors for the same analyte, wherein at least one of said sensors measures a fluid sample that is more concentrated with respect to a target analyte than the fluid sample measured by at least one other of said sensors.

In some embodiments, the relative wicking pressure capabilities of the different components may be used to control fluid sample concentration, to control fluid flow through the device, and/or remove unwanted solutes from the vicinity of the analyte sensors 2020, 2022, 2024. In such embodiments, the concentrator pump has a relative draw pressure capability 10 times that of the fluid collector 2032, and the fluid sample pump has a relative draw pressure capability 100 times that of the fluid collector 2032. In such embodiments, the external surface of the fluid collector is partially sealed so that fluid exchange can only occur between internal components in fluidic communication with each other.

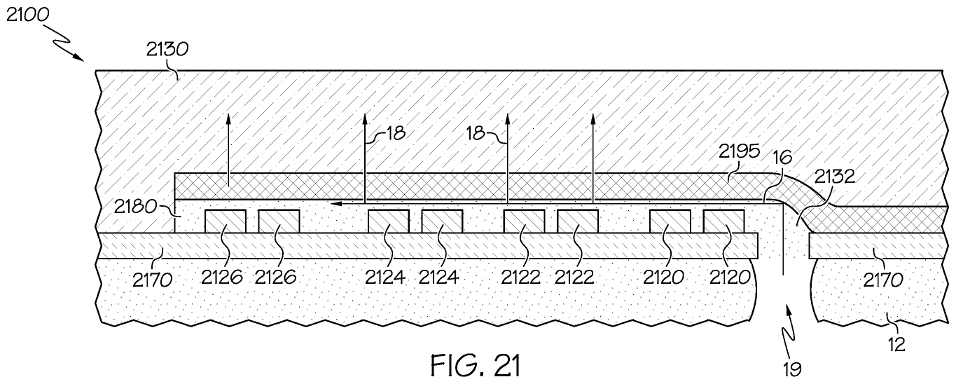

With reference to FIG. 21, wherein like numerals refer to like features of previous figures, a device 2100 is configured to concentrate a fluid sample extracted through a perforation or opening 19 in the skin 12. Perforation 19 may be formed by a laser, retractable needle, or other suitable means that allows for the extraction of an interstitial fluid sample. In at least one exemplary embodiment, the perforation 19 has a diameter between 10-100 μm or larger. A channel 2180 is filled with the sample fluid between a sample concentrator membrane 2195 and a fluid impermeable substrate or film 2170. The channel 2180 includes one or more analyte-specific sensors or sensor pairs (three sensor pairs, 2120, 2122, 2124, 2126 are shown). In the depicted embodiment, a coupler 2132 facilitates extraction of the interstitial fluid sample and draws the fluid in the direction indicated by the arrow 16. Coupler 2132 may be a wicking collector or coupler comprised of materials similar to, or the same as, wicking components previously described herein. Alternatively, interstitial fluid may be moved through opening 19 by pressure applied to the skin 12. This pressure could, for example, be applied by stretching the surface of the skin and maintaining the skin in a stretched condition. The skin could be maintained in a stretched condition by adhering the device to the stretched skin with, e.g., an adhesive on substrate 2170. As interstitial fluid flows through the channel 2180 in the direction of the arrow 16, water 18 from the fluid diffuses by osmosis or wicks into the sample pump 2130, which may be a hydrogel, a desiccant, salt, or microfluidic wicking material. By loss of water through the membrane, the fluid sample becomes more concentrated as it moves through the channel 2180. In embodiments with a plurality of analyte-specific sensors for detecting at least one target analyte, wherein at least two of the sensors comprise a first sensor group 2120, and at least two of the sensors comprise a second sensor group 2124, the first sensor group can measure the target analyte in the fluid sample when it is less concentrated, while the second sensor group measures the target analyte in the fluid when it is more concentrated.

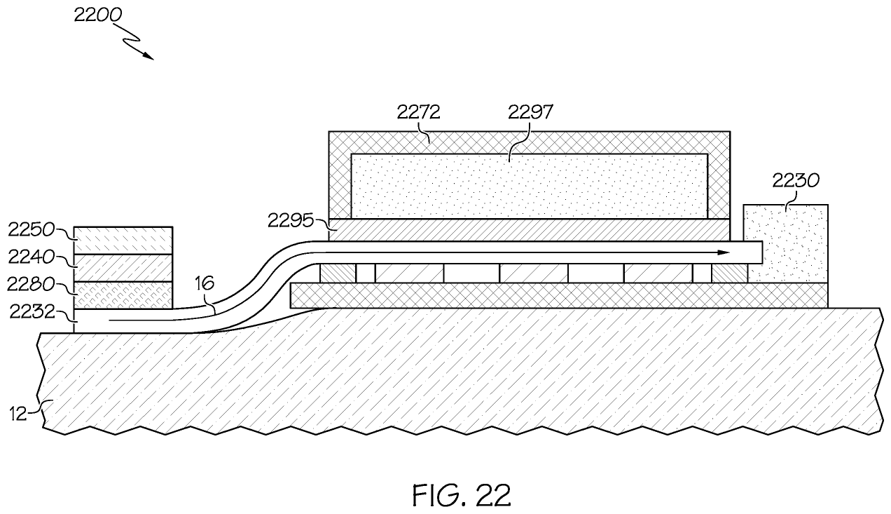

Turning now to FIG. 22, which is a variant of the previously taught device of FIG. 13A, a device 2200 may include electroosmosis and/or reverse iontophoresis capabilities for extracting interstitial fluid. For example, component 2250 could be an electrode, and component 2240 a gel containing a buffer solution to help neutralize or absorb pH change caused by the electrode 2250. Component 2280 is a membrane suitable for retaining the buffer solution in component 2240, but which passes an electrical current (ions). Device 2200 is capable of extracting a fluid like interstitial fluid from the skin using electro-osmosis (or reverse iontophoresis). The extracted fluid is received by a fluid collector 2232 and transported into the device. The extracted fluid is concentrated as the fluid flows through the collector 2232 in the direction of the arrow 16, and adjacent to a concentrator membrane 2295 and a concentrator pump material 2297 as described above, to facilitate detection of one or more target analytes in the fluid.

The following examples are provided to help illustrate the disclosed invention, and are not comprehensive or limiting in any manner. These examples serve to illustrate that although the specification herein does not list all possible device features or arrangements or methods for all possible applications, the invention is broad and may incorporate other useful methods or aspects of materials, devices, or other embodiments for the broad applications of the disclosed invention.

### Example 1

This example provides additional embodiments of membranes suitable for the disclosed invention, including calculations of criteria related to membrane operation in the invention. Membranes may utilize any material or filtration technique known by those skilled in the art of sample concentration or microfiltration. Solutes or analytes may be small ions, ions, small molecules, proteins, DNA, RNA, micro RNA or DNA, peptides, lipids, or any other solute or analyte of interest in biofluids. Commercially available ultrafiltration and filtration membranes are most effective for larger solutes found in biofluids, like proteins or peptides. Smaller molecules, including hormones and nucleotides, however, present a challenge, as they will typically pass through such membranes. Furthermore, if a membrane is used to block small molecules, but pass only water, then the concentration of salts, lactic acid, and other biofluid-abundant analytes could fall out of solution or hinder proper device or sensor performance. Other options, such as aquaporin and other lipid membranes, perform no better with small molecules that are lipophilic, and further tend to have limited shelf-lives caused by a tendency to dry out unless stored wet, among other things. Embodiments capable of sampling smaller biofluid analytes may therefore employ a membrane capable of forward osmosis (FO). Examples include a cellulose triacetate filter, like those produced by Hydration Technology Innovations; or the Dow Filmtec™ NF90-4040, a composite membrane made up of a polyamide active layer and a polysulfonic supporting layer, which works at low operating pressures. See A. Alturki, et al., “Removal of trace organic contaminants by the forward osmosis process” Separation and Purification Technology, 103 (2013) 258-266.

Such membranes can pass lactic acid (lactate), which is electrically charged and only 90 g/mole, or urea at 60 g/mole, as well as numerous salts. These solutes may be found at higher concentrations, so that if a biofluid sample, e.g., sweat, were concentrated 100×, these solute concentrations would correspondingly increase to the 1 M range, which could hinder device performance. Therefore, having a membrane that can concentrate the biofluid sample while allowing abundant solutes to pass through is advantageous. Further, the membrane must have high rejection rates for solutes of interest. For example, a small molecule like cortisol is uncharged, hydrophobic, and ˜362 g/mole, and therefore would be substantially rejected by the membrane and concentrated in the biofluid sample to be analyzed.

When operated in FO mode, i.e., with the membrane's dense side facing the biofluid sample to be concentrated, or feeder solution, and the membrane's porous side facing the concentrated draw solution, these materials are capable of processing a ˜1 M NaCl solution with a flux near 200 nL/min/mm₂. If the sensor device's microfluidic channel were 20 μm wide, each 1 mm₂ of that channel would have a biofluid volume of 20E-4 cm·0.1 cm·0.1 cm=2E-5 mL or 20 nL. Therefore, to achieve a sample concentration of 10×, the device would require, at most, a biofluid flow rate of approximately 20 nL/min/mm₂. If, through the use of lower biofluid volumes, the device was capable of fast biofluid sampling rates, e.g., every 5 minutes, then only 4 nL/min/mm₂ of biofluid would be required. Sweat generation rates in this range would allow concentration to occur at very low osmotic draw pressures, eliminating or reducing the need to augment draw pressures through the addition of a sugar (sucrose or glucose), or a salt, such as MgSO₄, to the draw solution.

While having a low osmotic pressure is desirable from a biofluid flow rate standpoint, osmotic pressure across the membrane still must be greater than the wicking pressure provided by biofluid collecting components, otherwise, the water in biofluid would not pass through the membrane. From A. Alturki, et al., osmotic pressure for a 0.5 M NaCl solution (with van't Hoff factor of 2) would be as follows: II=iMRT=2·(0.5 mol/L)(0.0821 L atm/mol/K)(298 K)=24.5 atm. Similarly, osmotic pressure for 0.5 M sucrose solution (with van't Hoff factor of 1) would be: II=MRT=1·(0.5 mol/L)(0.0821 L atm/mol/K)(298 K)=12.2 atm. To calculate the osmotic pressure achieved by adding saturated sucrose to drive biofluid across the membrane, the sucrose solubility limit in water is 2000 g/L/(342.30 g/mol)=5.8 mol/L or 5.8 M. Therefore, adding sucrose would provide osmotic pressures of around 141 atm or 101,000 N/m₂. Typical wicking pressures would be an order of magnitude lower. For example, pressure for a 20 μm high wicking channel (r=10 μM) would be (73E-3 N/m)/(10E-6 m)=7300 N/m₂ (14× less). Likewise, if using a 10×10 μm biofluid collector groove, the wicking pressures would be comparable to the 20 μm channel. Therefore, osmotic pressures for this embodiment of the invention would be sufficiently higher than wicking pressures to allow the FO membranes to function. Therefore, the invention may include a sample concentration component and at least one biofluid wicking component, where said concentration component has an osmotic pressure that is at least 2× greater and preferably 10× greater than wicking pressure of said wicking component.

If needed, draw pressures may also be augmented by adding capillary wicking pressure to the draw side of the membrane through use of microfluidics. Some embodiments may use osmotic pressure, wicking pressure, or a combination, to drive biofluid across the membrane, depending on the application. Therefore, the invention may also include a draw material that contains a wicking material that operates by capillary wicking pressure. Considerations determining the choice of method would include the need to drive biofluid abundant solutes, i.e., Na+, Cl− and K+, across the membrane to avoid fouling the concentrated biofluid sample. Also, biofluid sensor devices with larger biofluid volumes may require additional draw pressures to sense a given analyte. And certain biofluid applications may require or otherwise be limited to lower biofluid generation rates, which would also require higher draw pressures.

The above example can provide sample concentration for even challenging analytes such as cortisol (362 Da), especially if a similar analyte, i.e., cholesterol (387 Da) is also measured as a reference analyte, because it has a very low diurnal change (e.g., compare ratios of the two analytes). For example, if the membrane is cellulose acetate (which is very hydrophilic) lipophilic analytes such as cortisol could achieve 70% to 95% rejection or even greater. The above example will remove water, and the above example can also remove Na+, Cl−, K+, lactate (90 Da), urea (60 Da), and other high-concentration analytes that might be undesirable if they were also concentrated in the biofluid sample. The above examples could work well with draw solutions that are monosaccharides or disaccharides (100's of Da). Amino acids are found in sweat up mM levels. Many amino acids are small, and will readily pass through the disclosed concentration membranes. Assume average of 0.1 g/mL solubility limit, and average 100 g/mol. The molar concentration is 0.1×1000 g/L/(100 g/mol)=1 mol/L or 1M. Therefore, sweat could be concentrated by nearly 1000× before amino acids would precipitate out of sweat.

### Example 2

This example provides additional membrane embodiments suitable for the disclosed invention, including in some cases calculations of criteria related to their operation. More specifically, this example teaches an exemplary case for a determined amount of concentration as taught for FIG. 10. Assume a hydrophilic channel that is 7 μm tall by 500 μm wide and has a wicking pressure of ˜20,000 N/m₂. Assume a membrane that is biologically inert and ultra-pure, such as Biotech Cellulose Ester (CE) membranes, which offer a large range of concise molecular weight cut-offs (100 Da to 1,000,000 Da) and that tolerates weak or dilute acids, bases, and mild alcohols. Assume a molecular weight cut-off of ˜500 Da, and a concentrator pump with a draw material that is 7 mM of polyethyleneimine in water and/or other suitable solvent with a molecular weight of ˜10,000 Da. The draw solution may also contain other solutes found in natural biofluid (pH, salts, etc.) that may be desirable for proper sensor function or for other purposes. If each monomer of polyethyleneimine, which is a polyelectrolyte at pH<10, has a molecular weight of ˜50 Da, then there are ˜200 positive charges, and thus 200 counter-ions, likely chloride at pH values relevant to sweat (assume pH 6.5). Assuming full disassociation at the pHs observed in sweat, this draw solution would yield an osmotic pressure against natural sweat equivalent to about 10× greater than the osmotic pressure that sweat can generate. Therefore, the sweat will be concentrated about 10× for solutes that are >500 Da in size, and for the numerous solutes <500 Da they will largely be absorbed into the draw material through the membrane.

However, for continuous operation this would generally require the volume of the draw material to be very large compared to the total biofluid sample collected (otherwise the osmotic pressure difference will degrade over time). For example, the volume of the draw material could be 2× or 10× greater than the total biofluid sample volume collected, and more preferably >100× or even >1000×. Polyethyleneimine is not a natural solute in sweat. The disclosed invention may also therefore provide a determined amount of sample concentration, where the total osmolality of the concentrator pump is at least 2× greater than the total osmolality of biofluid. Still, a question remains as to how the osmolality differences between polyethyleneimine draw solution and natural sweat can be determined, because if the osmolality difference is not determined, then the amount of concentration occurring is more difficult to directly predict unless some other prediction method (including those taught herein) is utilized.

One example method which would work with multiple figures and embodiments of the invention, would be to have at least one sensor which measures the total osmolality of the natural biofluid coming into the device, using methods such as measuring total electrical conductance of biofluid, or by having a common pressure sensor which is surrounded (covered) by a membrane which passes mainly water and with an internal draw solution or material which therefore causes a pressure sensor to directly measure osmotic pressure and therefore osmolality of biofluid. For even greater precision, especially if the osmolality of the draw material/solution changes over time, such types of osmolality sensors may also be placed in the concentrator pump.

This has been a description of the disclosed invention along with a preferred method of practicing the disclosure, however the invention itself should only be defined by the appended claims.

What is claimed is:

1. A sensing device, comprising:
a target sensor for measuring a characteristic of a target analyte in a sample of a biofluid;
a collector for collecting and transporting the biofluid sample to the target sensor; and
a sample concentrator configured to generate a concentrated form of the biofluid sample to increase a first molarity of the target analyte to a second molarity, wherein the second molarity is at least two times higher than the first molarity.

2. The sensing device of claim 1, further comprising:
a reference sensor for measuring a reference analyte in the biofluid sample, wherein the sample concentrator is further configured to concentrate the biofluid sample to increase a third molarity of the reference analyte to a fourth molarity, wherein the fourth molarity is at least two times higher than the third molarity.

3. The sensing device of claim 2, wherein a ratio of the first molarity to the second molarity is substantially equal to a ratio of the third molarity to the fourth molarity.

4. The sensing device of claim 1, the sample concentrator further comprising:
a membrane that is permeable to water and impermeable to the target analyte, the membrane having a first surface adjacent to the biofluid sample and a second surface opposite the first surface.

5. The sensing device of claim 4, the sample concentrator further comprising:
a concentrator pump that exerts a force to move water or one or more solutes through the membrane and out of the biofluid sample to concentrate the biofluid sample relative to the target analyte.

6. The sensing device of claim 4, further comprising:
a draw material adjacent to, and in fluidic communication with, the second surface of the membrane; and
an osmolality sensor configured to measure an osmolality of the draw material.

7. The sensing device of claim 1, further comprising:
a flow-rate sensor for measuring a flow rate of the biofluid sample or a flow rate of the concentrated form of the biofluid sample.

8. The sensing device of claim 1, further comprising a plurality of target sensors comprising a first target sensor for measuring a characteristic of the target analyte at the first molarity and a second target sensor for measuring a characteristic of the target analyte at the second molarity.

9. The sensing device of claim 8, wherein the second target sensor has a dynamic range configured for use on a biofluid sample having a higher concentration than a dynamic range of the first target sensor.

10. The sensing device of claim 1, further comprising:
an osmolality sensor configured to measure a total osmolality of the biofluid sample.

11. The sensing device of claim 1, further comprising: a reverse iontophoresis component, comprising an electrode, a gel containing a solution for adjusting a potential of hydrogen value of the biofluid sample, and a membrane, wherein the membrane is in fluidic communication with the collector, and the gel is located between the membrane and the electrode.

12. The sensing device of claim 1, further comprising: a plurality of microneedles configured to pierce a skin surface and allow the biofluid sample to be in fluidic communication with the collector.

13. The sensing device of claim 1, further comprising a wicking collector configured to move a fluid sample to be in fluidic communication with the collector.

14. A method of using the sensing device of claim 1, the method comprising:
receiving a biofluid sample, wherein the biofluid sample is in fluidic communication with the sensing device;
generating a first concentrated biofluid sample by concentrating the biofluid sample with respect to a target analyte;
receiving, using the target sensor, a first measurement of the target analyte in the first concentrated biofluid sample, wherein the first measurement indicates a characteristic of the target analyte.

15. The method of claim 14, further comprising:
correlating the first measurement with a physiological condition associated with a source of the biofluid sample.

16. The method of claim 14, further comprising:
receiving, using a flow sensor, a measurement that indicates a flow rate of the biofluid sample; and
using the flow rate to estimate a concentration increase of the biofluid sample with respect to the target analyte.

17. The method of claim 14, further comprising:
generating a second concentrated biofluid sample by concentrating the first concentrated biofluid sample with respect to a reference analyte;
receiving, using a reference sensor, a second measurement of the reference analyte in the second concentrated biofluid sample, wherein the second measurement indicates a characteristic of the reference analyte; and
comparing the first measurement to the second measurement to estimate a concentration increase of the biofluid sample with respect to the target analyte.

18. The method of claim 14, further comprising:
receiving, using the target sensor, a third measurement associated with the target analyte prior to generating the first concentrated biofluid sample, wherein the third measurement indicates a characteristic of the target analyte;
comparing the first measurement to the third measurement;
estimating, based on comparing the first measurement and the third measurement, a flow rate of the biofluid sample; and
using the flow rate to estimate a concentration increase of the biofluid sample with respect to the target analyte.
